(19) United States
(12) Patent Application Publication
(10) Pub. No.: US 2016/0296513 A1
(43) Pub. Date: Oct. 13, 2016

(54) TREATMENT OF MULTIPLE SCLEROSIS BY ALEMTUZUMAB INDUCTION FOLLOWED BY LAQUINIMOD THERAPY

(71) Applicant: Volker Knappertz, Potomac, MD (US)

(72) Inventor: Volker Knappertz, Potomac, MD (US)

(73) Assignee: Teva Pharmaceutical Industries Ltd., Petach Tikva (IL)

(21) Appl. No.: 15/190,027

(22) Filed: Jun. 22, 2016

Related U.S. Application Data
(63) Continuation of application No. 14/909,042, filed on Jan. 29, 2016.
(60) Provisional application No. 61/920,085, filed on Dec. 23, 2013.
(60) Provisional application No. 61/861,354, filed on Aug. 1, 2013.

(51) Int. Cl. A61K 31/4704; C07K 16/28; A61K 45/06; A61K 9/28; A61K 39/395; A61K 9/00

(52) U.S. Cl. CPC A61K 31/4704; A61K 39/3955; A61K 9/0053; A61K 45/06; A61K 9/28; C07K 16/2893; A61K 2039/505

(57) ABSTRACT

This invention provides a method of treating a subject afflicted with multiple sclerosis (MS) or presenting clinically isolated syndrome (CIS) which comprises a) administering to the subject an amount of an anti-CD52 antibody, followed by b) periodically administering to the subject an amount of laquinimod. This invention also provides packages comprising pharmaceutical compositions of laquinimod or an anti-CD52 antibody for treating such a subject wherein laquinimod is to be administered as a maintenance therapy in such a subject who has received an anti-CD52 antibody induction therapy.

This application claims priority of U.S. Provisional Application No. 61/861,354, filed on Aug. 1, 2013, and U.S. Provisional Application No. 61/920,085, filed Dec. 23, 2013, the entire content of each of which is hereby incorporated by reference herein.

Throughout this application, various publications are referred to by first author and year of publication. Full citations for these publications are presented in a References section immediately before the claims. Disclosures of the documents and publications referred to herein are hereby incorporated in their entireties by reference into this application.

### BACKGROUND

Multiple Sclerosis (MS) is a neurological disease affecting more than 1 million people worldwide. It is the most common cause of neurological disability in young and middle-aged adults and has a major physical, psychological, social and financial impact on subjects and their families, friends and bodies responsible for health care (EMEA Guideline, 2006).

A clinically isolated syndrome (CIS) is a single monosymptomatic attack suggestive of MS, such as optic neuritis, brain stem symptoms, and partial myelitis. Patients with CIS that experience a second clinical attack are generally considered to have clinically definite multiple sclerosis (CDMS). Various MS disease stages and/or types are described in Multiple Sclerosis Therapeutics (Duntiz, 1999). Among them, relapsing-remitting multiple sclerosis (RRMS) is the most common form at the time of initial diagnosis. Many subjects with RRMS have an initial relapsing-remitting course for 5-15 years, which then advances into the secondary progressive MS (SPMS) disease course. There are currently a number of disease-modifying medications approved for use in relapsing MS (RMS), which includes RRMS and SPMS (The Disease Modifying Drug Brochure, 2006). These include interferon beta 1-a (Avonex® and Rebif®), interferon beta 1-b (Betaseron®), glatiramer acetate (Copaxone®), mitoxantrone (Novantrone®), natalizumab (Tysabri®) and Fingolimod (Gilenya®). Immunosuppressants or cytotoxic agents are used in some subjects after failure of conventional therapies. However, the relationship between changes of the immune response induced by these agents and the clinical efficacy in MS is far from settled (EMEA Guideline, 2006).

Other therapeutic approaches include symptomatic treatment which refers to all therapies applied to improve the symptoms caused by the disease (EMEA Guideline, 2006) and treatment of acute relapses with corticosteroids. While steroids do not affect the course of MS over time, they can reduce the duration and severity of attacks in some subjects.

Alemtuzumab

Alemtuzumab, marketed in the United States as Campath® and Europe as MabCampth®, is a recombinant DNA-derived humanized monoclonal IgGlx antibody raised against CD52, a 21-28 kD cell surface glycoprotein that is attached to the outer layer of the cell membrane by a glycosylphosphatidylinositol lipid anchor. CD52 is an abundant molecule (approximately 5×10⁵ antibody binding sites per cell) present on at least 95% of all human peripheral blood lymphocytes and monocytes/macrophages (Buttman and Rieckmann 2008, U.S. Patent Application Publication No. 2003-0108625).

Alemtuzumab is disclosed in U.S. Pat. No. 5,846,534, wherein a humanized antibody which binds effectively to the antigen CD52 as well as a method of treating a human patient having a lymphoid malignancy with such an antibody is described. Procedures for preparation and testing of such an antibody are disclosed (U.S. Pat. No. 5,846,534 and U.S. Patent Application Publication No. 2003-0108625, each of which is hereby incorporated by reference in its entireties into this application.).

Alemtuzumab was approved in 2001 in the U.S. and Europe for the treatment of fludarabine-refractory B-cell chronic lymphocytic leukemia (CLL) (Buttman and Rieckmann 2008 and U.S. Patent Application Publication No. 2003-0108625). As labeled for treatment of CLL, Campath therapy is initiated at a dose of 3 mg administered as a 2 hour i.v. infusion daily. When the Campath 3 mg daily dose is tolerated, the daily dose is escalated to 10 mg and continued until tolerated. When the mg dose is tolerated, the maintenance dose of Campath 30 mg/day is administered 3 times per week on alternate days (e.g., Monday, Wednesday, and Friday) for up to 12 weeks (see Campath® package insert, U.S. Patent Application Publication No. 2003-0108625).

In 2012 alemtuzumab was withdrawn from the U.S. and European market to prepare for launch under a different trade name, Lemtrada, for the treatment of multiple sclerosis (McKee 2012, Coles 2013). Two phase III clinical trials were conducted to investigate alemtuzumab in the treatment of multiple sclerosis with active comparator interferon beta-1a (Cole 2013).

The CARE-MS I Trial investigated the efficacy and safety of alemtuzumab as a treatment for relapsing-remitting multiple sclerosis (RRMS) in comparison with Rebif® (interferon beta-1a). The regimen of alemtuzumab used in CARE-MS I was 12 mg/day by intravenous (IV) injection for 5 consecutive days at Month 0 and 12 mg/day IV once a day for 3 consecutive days at Month 12. The regimen of interferon beta-1a used in CARE-MS I was 44 meg administered 3-times weekly by subcutaneous (SC) injection for 2 years (CARE-MS I, ClinicalTrials.gov and Cohen et al. 2012). Alemtuzumab was shown in CARE-MS I to provide reduction of relapse but did not show benefit in terms of disability endpoints (Cohen et al. 2012).

The CARE-MS II Trial investigated the efficacy and safety of two different doses of alemtuzumab for treatment of RRMS in comparison with Rebif® (interferon beta-1a). The regimens of alemtuzumab used in CARE-MS II were 12 and 24 mg/day IV for 5 consecutive days at Month 0 and 12 and 24 mg/day IV once a day for 3 consecutive days at Month 12. The regimen of interferon beta-1a used in CARE-MS II was the same as in CARE-MS I (CARE-MS II, ClinicalTrials.gov and Coles et al. 2012). In CARE-MS II it was found that alemtuzumab could be used to reduce relapse rates and sustain accumulation of disability in patients with first-line treatment-refractory RRMS (Coles 2012).

Treatment of multiple sclerosis using alemtuzumab was described in, e.g., U.S. Pat. No. 6,120,766, U.S. Application Publication No. 2013-0108625 and European Application Publication Nos. EP243364 9 A2, EP2066352 A1 and EP2444104 A3, each of which is hereby incorporated by reference in its entireties into this application.

Laquinimod

Laquinimod (TV-5600) is a novel synthetic compound with high oral bioavailability which has been suggested as an oral formulation for the treatment of Multiple sclerosis (MS) (Polman, 2005; Sandberg-Wollheim, 2005; Comi et al 2008). Laquinimod and its sodium salt form are described, for example, in U.S. Pat. No. 6,077,851. The mechanism of action of laquinimod is not fully understood.

Animal studies show it causes a Th1 (T helper 1 cell, produces pro-inflammatory cytokines) to Th2 (T helper 2 cell, produces anti-inflammatory cytokines) shift with an anti-inflammatory profile (Yang, 2004; Brück, 2011). Another study demonstrated (mainly via the NFkB pathway) that laquinimod induced suppression of genes related to antigen presentation and corresponding inflammatory pathways (Gurevich, 2010). Other suggested potential mechanisms of action include inhibition of leukocyte migration into the CNS, increase of axonal integrity, modulation of cytokine production, and increase in levels of brain-derived neurotrophic factor (BDNF) (Runström, 2006; Brück, 2011).

Laquinimod showed a favorable safety and tolerability profile in two phase III trials (Results of Phase III BRAVO Trial Reinforce Unique Profile of Laquinimod for Multiple Sclerosis Treatment; Teva Pharma, Active Biotech Post Positive Laquinimod Phase 3 ALLEGRO Results).

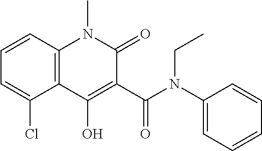 [SMILES: CCN(C(=O)c1c(O)c2c(Cl)cccc2n(C)c1=O)c1ccccc1]

IUPAC: 5-chloro-N-ethyl-4-hydroxy-1-methyl-2-oxo-N-phenyl-1,2-dihydroquinoline-3-carboxamide

Combination Therapy

The administration of two drugs to treat a given condition, such as multiple sclerosis, raises a number of potential problems. In vivo interactions between two drugs are complex. The effects of any single drug are related to its absorption, distribution, and elimination. When two drugs are introduced into the body, each drug can affect the absorption, distribution, and elimination of the other and hence, alter the effects of the other. For instance, one drug may inhibit, activate or induce the production of enzymes involved in a metabolic route of elimination of the other drug (Guidance for Industry, 1999). In one example, combined administration of fingolimod and interferon (IFN) has been experimentally shown to abrogate the clinical effectiveness of either therapy. (Brod 2000) In another experiment, it was reported that the addition of prednisone in combination therapy with IFN-β antagonized its up-regulator effect. Thus, when two drugs are administered to treat the same condition, it is unpredictable whether each will complement, have no effect on, or interfere with, the therapeutic activity of the other in a human subject.

Not only may the interaction between two drugs affect the intended therapeutic activity of each drug, but the interaction may increase the levels of toxic metabolites (Guidance for Industry, 1999). The interaction may also heighten or lessen the side effects of each drug. Hence, upon administration of two drugs to treat a disease, it is unpredictable what change will occur in the negative side profile of each drug. In one example, the combination of natalizumab and interferon β-1a was observed to increase the risk of unanticipated side effects. (Vollmer, 2008; Rudick 2006; Kleinschmidt-DeMasters, 2005; Langer-Gould 2005)

Additionally, it is difficult to accurately predict when the effects of the interaction between the two drugs will become manifest. For example, metabolic interactions between drugs may become apparent upon the initial administration of the second drug, after the two have reached a steady-state concentration or upon discontinuation of one of the drugs (Guidance for Industry, 1999).

Therefore, the state of the art at the time of filing is that the effects of combination therapy of two drugs, in particular the combination of an anti-CD52 antibody, e.g., alemtuzumab induction therapy with laquinimod maintenance therapy, cannot be predicted.

### SUMMARY OF THE INVENTION

The subject invention provides a method of treating a subject afflicted with multiple sclerosis or presenting a clinically isolated syndrome and who has received an anti-CD52 antibody induction therapy, comprising periodically administering to the subject an amount of laquinimod, thereby treating the subject afflicted with multiple sclerosis or presenting a clinically isolated syndrome.

The subject invention provides a method of treating a subject afflicted with multiple sclerosis or presenting a clinically isolated syndrome comprising a) administering to the subject an amount of an anti-CD52 antibody, followed by b) periodically administering to the subject an amount of laquinimod, thereby treating the subject afflicted with multiple sclerosis or presenting a clinically isolated syndrome.

The subject invention also provides a package comprising: a) a first pharmaceutical composition comprising an amount of laquinimod and a pharmaceutically acceptable carrier; b) a second pharmaceutical composition comprising an amount of an anti-CD52 antibody and a pharmaceutically acceptable carrier; and c) instructions for use of the first and, second pharmaceutical compositions together to treat a subject afflicted with multiple sclerosis or presenting a clinically isolated syndrome.

The subject invention also provides laquinimod for use as a maintenance therapy in treating a subject afflicted with multiple sclerosis or presenting a clinically isolated, syndrome, wherein the subject has received an anti-CD52 antibody induction therapy.

The subject invention also provides a pharmaceutical composition comprising an amount of laquinimod for use as a maintenance therapy in treating a subject afflicted with multiple sclerosis or presenting a clinically isolated syndrome, wherein the subject has received an anti-CD52 antibody induction therapy.

The subject invention also provides a pharmaceutical composition comprising an amount of laquinimod for use in treating a subject afflicted, with multiple sclerosis or presenting a clinically isolated syndrome as a maintenance therapy in combination with an anti-CD52 antibody induction therapy.

The subject invention also provides a pharmaceutical composition comprising an amount of an anti-CD52 antibody for use in treating a subject afflicted with multiple sclerosis or presenting a clinically isolated syndrome as an induction therapy in combination with laquinimod maintenance therapy.

The subject invention also provides a therapeutic package for dispensing to, or for use in dispensing to, a subject afflicted with multiple sclerosis or presenting a clinically isolated syndrome, which comprises: a) one or more unit doses, each such unit dose comprising: i) an amount of laquinimod and/or ii) an amount of an anti-CD52 antibody, and b) a finished pharmaceutical container therefor, said container containing said unit dose or unit doses, said container further containing or comprising labeling directing the use of said package in the treatment of said subject.

### DETAILED DESCRIPTION OF THE INVENTION

The subject invention provides a method of treating a subject afflicted with multiple sclerosis or presenting a clinically isolated syndrome and who has received an anti-CD52 antibody induction therapy, comprising periodically administering to the subject an amount of laquinimod, thereby treating the subject afflicted with multiple sclerosis or presenting a clinically isolated syndrome. In one embodiment, the method consists essentially of periodically administering to the subject an amount of laquinimod.

In one embodiment, the method further comprises a step of determining that the subject has received the anti-CD52 antibody induction therapy. In a particular embodiment, the method further comprises a step of determining that the subject has received the anti-CD52 antibody induction therapy prior to administration of laquinimod.

The subject invention provides a method of treating a subject afflicted with multiple sclerosis or presenting a clinically isolated syndrome comprising a) administering to the subject an amount of an anti-CD52 antibody, followed by b) periodically administering to the subject an amount of laquinimod. In one embodiment, the method consists essentially of a) administering to the subject an amount of an anti-CD52 antibody, followed by b) periodically administering to the subject an amount of laquinimod.

In one embodiment, the multiple sclerosis is relapsing multiple sclerosis. In another embodiment, the relapsing multiple sclerosis is relapsing-remitting multiple sclerosis.

In one embodiment, step a) comprises periodically administering to the subject an amount of the anti-CD52 antibody. In another embodiment, step a) followed by step b) is more effective to treat the subject then step a) alone or step b) alone.

In an embodiment, step a) followed by step b) is effective to reduce a symptom of multiple sclerosis in the subject. In another embodiment, the symptom is a MRI-monitored multiple sclerosis disease activity, relapse rate, accumulation of physical disability, frequency of relapses, decreased time to confirmed disease progression, decreased time to confirmed relapse, frequency of clinical exacerbation, brain atrophy, neuronal dysfunction, neuronal injury, neuronal degeneration, neuronal apoptosis, risk for confirmed progression, deterioration of visual function, fatigue, impaired mobility, cognitive impairment, reduction of brain volume, abnormalities observed in whole Brain MTR histogram, deterioration in general health status, functional status, quality of life, and/or symptom severity on work.

In one embodiment, step a) followed by step b) is effective to decrease or inhibit reduction of brain volume. In another embodiment, brain volume is measured by percent brain volume change (PBVC).

In one embodiment, step a) followed by step b) is effective to increase time to confirmed disease progression. In another embodiment, time to confirmed disease progression is increased by 20-60%. In yet another embodiment, time to confirmed disease progression is increased by 50%.

In one embodiment, step a) followed by step b) is effective to decrease abnormalities observed in whole Brain MTR histogram. In another embodiment, the accumulation of physical disability is measured by Kurtzke Expanded Disability Status Scale (EDSS) score. In another embodiment, the accumulation of physical disability is assessed by the time to confirmed disease progression as measured by Kurtzke Expanded Disability Status Scale (EDSS) score.

In one embodiment, the subject had an EDSS score of 0-5.5 at baseline. In another embodiment, the subject had an EDSS score of 1.5-4.5 at baseline. In another embodiment, the subject had an EDSS score of 5.5 or greater at baseline. In another embodiment, confirmed disease progression is a 1 point increase of the EDSS score. In another embodiment, confirmed disease progression is a 0.5 point increase of the EDSS score.

In an embodiment, impaired mobility is assessed by the Timed-25 Foot Walk test. In another embodiment, impaired mobility is assessed by the 12-Item Multiple Sclerosis Walking Scale (MSWS-12) self-report questionnaire. In another embodiment, impaired mobility is assessed by the Ambulation Index (AI). In another embodiment, impaired mobility is assessed by the Six-Minute Walk (6MW) Test. In another embodiment, impaired mobility is assessed by the Lower Extremity Manual Muscle Test (LEMMT) Test. In one embodiment, step a) followed by step b) is effective to reduce cognitive impairment. In another embodiment, cognitive impairment is assessed by the Symbol Digit Modalities Test (SDMT) score.

In one embodiment, general health status is assessed by the EuroQoL (EQ5D) questionnaire, Subject Global Impression (SGI) or Clinician Global Impression of Change (CGIC). In another embodiment, functional status is measured by the subject's Short-Form General Health survey (SF-36) Subject Reported Questionnaire score. In another embodiment, quality of life is assessed by SF-36, EQ5D, Subject Global Impression (SGI) or Clinician Global Impression of Change (CGIC). In another embodiment, the subject's SF-36 mental component summary score (MSC) is improved. In another embodiment, the subject's SF-36 physical component summary sore (PSC) is improved. In another embodiment, fatigue is assessed by the EQ5D, the subject's Modified Fatigue Impact Scale (MFIS) score or the French valid versions of the Fatigue Impact Scale (EMIF-SEP) score. In another embodiment, symptom severity on work is measured by the work productivity and activities impairment General Health (WPAI-GH) questionnaire.

In an embodiment, laquinimod is laquinimod sodium. In another embodiment, the laquinimod is administered via oral administration. In another embodiment, the anti-CD52 antibody is/was administered via injection. In another embodiment, the anti-CD52 antibody is/was administered via intravenous injection. In one embodiment, the laquinimod and/or the anti-CD52 antibody is administered daily. In another embodiment, the laquinimod and/or the anti-CD52 antibody is administered more often than once daily. In another embodiment, the laquinimod and/or the anti-CD52 antibody is administered less often than once daily.

In one embodiment, the amount laquinimod administered is less than 0.6 mg/day. In another embodiment, the amount laquinimod administered is 0.1-40.0 mg/day. In another embodiment, the amount laquinimod administered is 0.1-2.5 mg/day. In another embodiment, the amount laquinimod administered is 0.25-2.0 mg/day. In another embodiment, the amount laquinimod administered is 0.5-1.2 mg/day. In another embodiment, the amount laquinimod administered is 0.25 mg/day. In another embodiment, the amount laquinimod administered is 0.3 mg/day. In another embodiment, the amount laquinimod administered is 0.5 mg/day. In another embodiment, the amount laquinimod administered is 0.6 mg/day. In another embodiment, the amount laquinimod administered is 1.0 mg/day. In another embodiment, the amount laquinimod administered is 1.2 mg/day. In another embodiment, the amount laquinimod administered is 1.5 mg/day. In another embodiment, the amount laquinimod administered is 2.0 mg/day.

In one embodiment of the present invention, step b) begins after completion of step a). In another embodiment, step b) begins on the same day as initiation of step a). In yet another embodiment, step b) begins 1-10 days after initiation of step a).

In one embodiment, the method further comprises: c) administering to the subject an amount of the anti-CD52 antibody. In another embodiment, step c) begins after completion of step a). In another embodiment, step c) begins on the same day as initiation of step b). In another embodiment, step c) begins at least 6 months after initiation of step a). In another embodiment, step c) begins at least 12 months after initiation of step a). In another embodiment, step c) begins at least 18 months after initiation of step a). In another embodiment, step c) begins at least 24 months after initiation of step a). In another embodiment, step c) begins at least 18 months after initiation of step a). In another embodiment, step c) begins at 1-24 months after initiation of step a).

In one embodiment, the amount of the anti-CD52 antibody administered in step a) and/or step c) is 3-48 mg/day. In another embodiment, the amount of the anti-CD52 antibody administered in step a) and/or step c) is 9-36 mg/day. In another embodiment, the amount of the anti-CD52 antibody administered in step a) and/or step c) is 1-12 mg/day. In another embodiment, the amount of the anti-CD52 antibody administered in step a) and/or step c) is 2-10 mg/day. In another embodiment, the amount of the anti-CD52 antibody administered in step a) and/or step c) is 5-30 mg/day. In another embodiment, the amount of the anti-CD52 antibody administered in step a) and/or step c) is 10-25 mg/day. In another embodiment, the amount of the anti-CD52 antibody administered in step a) and/or step c) is 2 mg/day. In another embodiment, the amount of the anti-CD52 antibody administered in step a) and/or step c) is 3 mg/day. In another embodiment, the amount of the anti-CD52 antibody administered in step a) and/or step c) is 10 mg/day. In another embodiment, the amount of the anti-CD52 antibody administered in step a) and/or step c) is 12 mg/day. In another embodiment, the amount of the anti-CD52 antibody administered in step a) and/or step c) is 18 mg/day. In another embodiment, the amount of the anti-CD52 antibody administered in step a) and/or step c) is 20 mg/day. In another embodiment, the amount of the anti-CD52 antibody administered in step a) and/or step c) is 24 mg/day. In another embodiment, the amount of the anti-CD52 antibody administered in step a) and/or step c) is 30 mg/day.

In another embodiment, the amount of the anti-CD52 antibody administered in step a) and/or step c) is suboptimal. In another embodiment, the amount of the anti-CD52 antibody administered in step a) and/or step c) is less than 5 mg/day. In another embodiment, the amount of the anti-CD52 antibody administered in step a) and/or step c) is less than 10 mg/day. In another embodiment, the amount of the anti-CD52 antibody administered in step a) and/or step c) is less than 12 mg/day. In another embodiment, the amount of the anti-CD52 antibody administered in step a) and/or step c) is less than 24 mg/day.

In an embodiment, step a) and/or step c) comprises daily administration of an amount of the anti-CD52 antibody to the subject over 1-5 days. In another embodiment, step a) and/or step c) comprises at least one administration of an amount of the anti-CD52 antibody to the subject over one day. In another embodiment, step a) and/or step c) comprises daily administration of an amount of the anti-CD52 antibody to the subject for at least 3 consecutive days. In another embodiment, step a) and/or step c) comprises daily administration of an amount of the anti-CD52 antibody to the subject for at least 5 consecutive days.

In another embodiment, step a) and/or step c) comprises 1-5 daily doses of the anti-CD52 antibody of which are administered on consecutive days, wherein the daily dose is >0 and ≦12 mg. In another embodiment, step a) and/or step c) comprises 1-10 doses of the anti-CD52 antibody of which are administered over a period of up to two weeks. In certain embodiments, the anti-CD52 antibody is administered at a dose of 11, 10, 9, 8, 7, 6, 5 or 4 mg/day for a period of 5 days. In other embodiments, the anti-CD52 antibody is administered at a dose of 11, 10, 9, 8, 7, 6, 5 or 4 mg/day for a period of 2, 3 or 4 days.

In an embodiment, step a) and/or step c) comprises administration of an amount of the anti-CD52 antibody on alternate days. In an embodiment, the duration of step a) and/or step c) is 1-12 days. In an embodiment, the duration of step a) and/or step c) is 5 days. In an embodiment, the duration of step a) and/or step c) is 12 days.

In an embodiment of any of the methods described herein, the method further comprises administration of nonsteroidal anti-inflammatory drugs (NSAIDs), salicylates, slow-acting drugs, gold compounds, hydroxychloroquine, sulfasalazine, combinations of slow-acting drugs, corticosteroids, cytotoxic drugs, immunosuppressive drugs and/or antibodies.

In one embodiment, the periodic administration of laquinimod (e.g., in step b)) continues for at least 3 days. In another embodiment, the periodic administration of laquinimod continues for more than 30 days. In another embodiment, the periodic administration of laquinimod continues for more than 42 days. In another embodiment, the periodic administration of laquinimod continues for 8 weeks or more. In another embodiment, the periodic administration of laquinimod continues for at least 12 weeks. In another embodiment, the periodic administration of laquinimod continues for at least 24 weeks. In another embodiment, the periodic administration of laquinimod continues for more than 24 weeks. In another embodiment, the periodic administration of laquinimod continues for 6 months or more.

In one embodiment, step a) followed by step b) inhibits a symptom of relapsing multiple sclerosis by at least 20%. In another embodiment, step a) followed by step b) inhibits a symptom of relapsing multiple sclerosis by at least 30%. In another embodiment, step a) followed by step b) inhibits a symptom of relapsing multiple sclerosis by at least 50%. In another embodiment, step a) followed by step b) inhibits a symptom of relapsing multiple sclerosis by at least 70%. In another embodiment, step a) followed by step b) inhibits a symptom of relapsing multiple sclerosis by more than 100%. In another embodiment, step a) followed by step b) inhibits a symptom of relapsing multiple sclerosis by more than 300%. In another embodiment, step a) followed by step b) inhibits a symptom of relapsing multiple sclerosis by more than 1000%. In an embodiment the comparison is to a patient at baseline. In another embodiment the comparison is to a patient at prior to receiving laquinimod.

In one embodiment, each of the amount of laquinimod or pharmaceutically acceptable salt thereof when taken alone, and the amount of the anti-CD52 antibody when taken alone is effective to treat the subject. In another embodiment, either the amount of laquinimod or pharmaceutically acceptable salt thereof when taken alone, the amount of the anti-CD52 antibody when taken alone, or each such amount when taken alone is not effective to treat the subject.

In one embodiment, the subject is a human patient.

The subject invention also provides a package comprising: a) a first pharmaceutical composition comprising an amount of laquinimod and a pharmaceutically acceptable carrier; b) a second pharmaceutical composition comprising an amount of an anti-CD52 antibody and a pharmaceutically acceptable carrier; and c) instructions for use of the first and second pharmaceutical compositions together to treat a subject afflicted with multiple sclerosis or presenting a clinically isolated syndrome. In one embodiment, the package consists essentially of components a), b) and c).

In one embodiment, the first pharmaceutical composition, the second pharmaceutical composition, or both the first and the second pharmaceutical composition are in an aerosol, an inhalable powder, an injectable, a liquid, a solid, a capsule or a tablet form. In another embodiment, the first pharmaceutical composition, the second pharmaceutical composition, or both the first and the second pharmaceutical composition are in a liquid or a solid form.

In an embodiment, the first pharmaceutical composition is in capsule form or in tablet form. In another embodiment, the tablets are coated with a coating which inhibits oxygen from contacting the core. In another embodiment, the coating comprises a cellulosic polymer, a detackifier, a gloss enhancer, or pigment.

In one embodiment, the first pharmaceutical composition further comprises mannitol. In another embodiment, the first pharmaceutical composition further comprises an alkalinizing agent. In another embodiment, the alkalinizing agent is meglumine. In another embodiment, the first pharmaceutical composition further comprises an oxidation reducing agent.

In one embodiment, the first pharmaceutical composition is stable and free of an alkalinizing agent or an oxidation reducing agent. In another embodiment, the first pharmaceutical composition is free of an alkalinizing agent and free of an oxidation reducing agent.

In an embodiment, the first pharmaceutical composition is stable and free of disintegrant. In another embodiment, the first pharmaceutical composition further comprises a lubricant. In another embodiment, the lubricant is present in the composition as solid particles. In yet another embodiment, the lubricant is sodium stearyl fumarate or magnesium stearate.

In one embodiment, the first pharmaceutical composition further comprises a filler. In another embodiment, the filler is present in the composition as solid particles. In another embodiment, the filler is lactose, lactose monohydrate, starch, isomalt, mannitol, sodium starch glycolate, sorbitol, lactose spray dried, lactose anhydrouse, or a combination thereof. In yet another embodiment, the filler is mannitol or lactose monohydrate.

In one embodiment of any of the packages described herein, the package further comprises a desiccant. In another embodiment, the desiccant is silica gel.

In one embodiment, the first pharmaceutical composition is stable and has a moisture content of no more than 4%. In another embodiment, laquinimod is present in the composition as solid particles. In another embodiment, the package is a sealed packaging having a moisture permeability of not more than 15 mg/day per liter. In another embodiment, the sealed package is a blister pack in which the maximum moisture permeability is no more than 0.005 mg/day. In another embodiment, the sealed package is a bottle. In another embodiment, the bottle is closed with a heat induction liner. In another embodiment, the sealed package comprises an HDPE bottle. In another embodiment, the sealed package comprises an oxygen absorbing agent. In another embodiment, the oxygen absorbing agent is iron.

In one embodiment, the amount of laquinimod in the first composition comprises a unit dose of less than 0.6 mg. In another embodiment, the amount of laquinimod in the first composition comprises a unit dose of 0.1-40.0 mg. In another embodiment, the amount of laquinimod in the first composition comprises a unit dose of 0.1-2.5 mg. In another embodiment, the amount of laquinimod in the first composition comprises a unit dose of 0.25-2.0 mg. In another embodiment, the amount of laquinimod in the first composition comprises a unit dose of 0.5-1.2 mg. In another embodiment, the amount of laquinimod in the first composition comprises a unit dose of 0.25 mg. In another embodiment, the amount of laquinimod in the first composition comprises a unit dose of 0.3 mg. In another embodiment, the amount of laquinimod in the first composition comprises a unit dose of 0.5 mg. In another embodiment, the amount of laquinimod in the first composition comprises a unit dose of 0.6 mg. In another embodiment, the amount of laquinimod in the first composition comprises a unit dose of 1.0 mg. In another embodiment, the amount of laquinimod in the first composition comprises a unit dose of 1.2 mg. In another embodiment, the amount of laquinimod in the first composition comprises a unit dose of 1.5 mg. In another embodiment, the amount of laquinimod in the first composition comprises a unit dose of 2.0 mg.

In one embodiment, the amount of the anti-CD52 antibody in the second composition comprises a unit dose of 3-48 mg. In another embodiment, the amount of the anti-CD52 antibody in the second composition comprises a unit dose of 9-36 mg. In another embodiment, the amount of the anti-CD52 antibody in the second composition comprises a unit dose of 5-30 mg. In another embodiment, the amount of the anti-CD52 antibody in the second composition comprises a unit dose of 10-25 mg. In another embodiment, the amount of the anti-CD52 antibody in the second composition comprises a unit dose of 1-12 mg. In another embodiment, the amount of the anti-CD52 antibody in the second composition comprises a unit dose of 12 mg. In another embodiment, the amount of the anti-CD52 antibody in the second composition comprises a unit dose of 24 mg. In another embodiment, the amount of the anti-CD52 antibody in the second composition comprises a unit dose of less than 12 mg. In another embodiment, the amount of the anti-CD52 antibody in the second composition comprises a unit dose of less than 24 mg.

The subject invention also provides laquinimod for use as a maintenance therapy in treating a subject afflicted with multiple sclerosis or presenting a clinically isolated syndrome, wherein the subject has received an anti-CD52 antibody induction therapy.

The subject invention also provides a pharmaceutical composition comprising an amount of laquinimod for use as a maintenance therapy in treating a subject afflicted with multiple sclerosis or presenting a clinically isolated syndrome, wherein the subject has received an anti-CD52 antibody induction therapy. In one embodiment, the pharmaceutical composition consists essentially of an amount of laquinimod.

In one embodiment, laquinimod is laquinimod sodium. In another embodiment, the pharmaceutical composition is in an aerosol, an inhalable powder, an injectable, a liquid, a solid, a capsule or a tablet form. In another embodiment, the pharmaceutical composition is in a liquid or a solid form. In another embodiment, the pharmaceutical composition is in capsule form or in tablet form. In another embodiment, the tablets are coated with a coating which inhibits oxygen from contacting the core. In yet another embodiment, the coating comprises a cellulosic polymer, a detackifier, a gloss enhancer, or pigment.

In one embodiment, the pharmaceutical composition further comprises mannitol. In another embodiment, the pharmaceutical composition further comprises an alkalinizing agent. In another embodiment, the alkalinizing agent is meglumine. In another embodiment, the pharmaceutical composition further comprises an oxidation reducing agent.

In one embodiment, the pharmaceutical composition is free of an alkalinizing agent or an oxidation reducing agent. In another embodiment, it is free of an alkalinizing agent and free of an oxidation reducing agent.

In one embodiment, the pharmaceutical composition is stable and free of disintegrant. In another embodiment, it further comprises a lubricant. In another embodiment, the lubricant is present in the composition as solid particles. In another embodiment, the lubricant is sodium stearyl fumarate or magnesium stearate.

In one embodiment, the pharmaceutical composition further comprises a filler. In another embodiment, the filler is present in the composition as solid particles. In another embodiment, the filler is lactose, lactose monohydrate, starch, isomalt, mannitol, sodium starch glycolate, sorbitol, lactose spray dried, lactose anhydrouse, or a combination thereof. In yet another embodiment, the filler is mannitol or lactose monohydrate.

In one embodiment, the amount of laquinimod in the composition is less than 0.6 mg. In another embodiment, the amount of laquinimod in the composition is 0.1-40.0 mg. In another embodiment, the amount of laquinimod in the composition is 0.1-2.5 mg. In another embodiment, the amount of laquinimod in the composition is 0.25-2.0 mg. In another embodiment, the amount of laquinimod in the composition is 0.5-1.2 mg. In another embodiment, the amount of laquinimod in the composition is 0.25 mg. In another embodiment, the amount of laquinimod in the composition is 0.3 mg. In another embodiment, the amount of laquinimod in the composition is 0.5 mg. In another embodiment, the amount of laquinimod in the composition is 0.6 mg. In another embodiment, the amount of laquinimod in the composition is 1.0 mg. In another embodiment, the amount of laquinimod in the composition is 1.2 mg. In another embodiment, the amount of laquinimod in the composition is 1.5 mg. In another embodiment, the amount of laquinimod in the composition is 2.0 mg.

The subject invention also provides a pharmaceutical composition comprising an amount of laquinimod for use in treating a subject afflicted with multiple sclerosis or presenting a clinically isolated syndrome as a maintenance therapy in combination with an an anti-CD52 antibody induction therapy. In an embodiment, the pharmaceutical composition consists essentially of an amount of laquinimod.

The subject invention also provides a pharmaceutical composition comprising an amount of an anti-CD52 antibody for use in treating a subject afflicted with multiple sclerosis or presenting a clinically isolated syndrome as an induction therapy in combination with laquinimod maintenance therapy. In an embodiment, the pharmaceutical composition consists essentially of an amount of an anti-CD52 antibody.

The subject invention also provides a therapeutic package for dispensing to, or for use in dispensing to, a subject afflicted with multiple sclerosis or presenting a clinically isolated syndrome, which comprises: a) one or more unit doses, each such unit dose comprising: i) an amount of laquinimod and/or ii) an amount of an anti-CD52 antibody, and b) a finished pharmaceutical container therefor, said container containing said unit dose or unit doses, said container further containing or comprising labeling directing the use of said package in the treatment of said subject.

In an embodiment, the therapeutic package consists essentially of a) one or more unit doses, each such unit dose comprising: i) an amount of laquinimod and/or ii) an amount of ah anti-CD52 antibody, and b) a finished pharmaceutical contained therefor, said container containing said unit dose or unit doses, said contained further containing or comprising labeling directing the use of said package in the treatment of said subject.

In a particular embodiment of the present invention, the respective amounts of said laquinimod and said anti-CD52 antibody in said unit dose when taken together is more effective to treat the subject than when compared to the administration of said laquinimod in the absence of said anti-CD52 antibody or the administration of said anti-CD52 antibody in the absence of said laquinimod.

The subject invention also provides a method of treating a subject afflicted with multiple sclerosis or presenting a clinically isolated syndrome comprising a) periodically administering to the subject an amount of an anti-CD52 antibody, and b) periodically administering to the subject an amount of laquinimod, wherein step a) and b) begins on the same day. In an embodiment, the method consists essentially of a) periodically administering to the subject an amount of an anti-CD52 antibody, and b) periodically administering to the subject an amount of laquinimod.

In an embodiment of any of the methods described herein, the method excludes administration of an active pharmaceutical agent which is not laquinimod or a CD52 specific antibody (an anti-CD52 antibody). In another embodiment, the method excludes administration of an active pharmaceutical agent which is not laquinimod or alemtuzumab.

In an embodiment of any of the packages or pharmaceutical compositions described herein, the package or pharmaceutical composition does not comprise any active pharmaceutical agent which is not laquinimod or the CD52 specific antibody (an anti-CD52 antibody, e.g., alemtuzumab).

In an embodiment of any one of the method, package, use, pharmaceutical composition, or therapeutic package described above, the anti-CD52 antibody binds to the same epitope as alemtuzumab. In another embodiment, the anti-CD52 antibody is alemtuzumab.

For the foregoing embodiments, each embodiment disclosed herein is contemplated as being applicable to each of the other disclosed embodiment. For example, the elements recited in the method embodiments can be used in the composition and package embodiments described herein and vice versa.

The anti-CD52 antibody, e.g., alemtuzumab can be administered by way of oral, sublingual, injection including subcutaneous, intramuscular and intravenous, topical, intratracheal, intranasal, transdermal or rectal administration. The anti-CD52 antibody may be administered in admixture with conventional pharmaceutical carriers. The appropriate unit forms of administration include forms for injection, e.g., intravenous administration.

Laquinimod mixtures, compositions, and the process for the manufacture thereof are described in, e.g., U.S. Pat. No. 6,077,851, U.S. Pat. No. 7,884,208, U.S. Pat. No. 7,989,473, U.S. Pat. No. 8,178,127, U.S. Application Publication No. 2010-0055072, U.S. Application Publication No. 2012-0010238, and U.S. Application Publication No. 2012-0010239, each of which is hereby incorporated by reference in its entireties into this application.

Use of laquinimod for treatment of various conditions, and the corresponding dosages and regimens, are described in U.S. Pat. No. 6,077,851 (multiple sclerosis, insulin-dependent diabetes mellitus, systemic lupus erythematosus, rheumatoid arthritis, inflammatory bowel disease, psoriasis, inflammatory respiratory disorder, atherosclerosis, stroke, and Alzheimer's disease), U.S. Application Publication No. 2011-0027219 (Crohn's disease), U.S. Application Publication No. 2010-0322900 (Relapsing-remitting multiple sclerosis), U.S. Application Publication No. 2011-0034508 (brain-derived neurotrophic factor (BDNF)-related diseases), U.S. Application Publication No. 2011-0218179 (active lupus nephritis), U.S. Application Publication No. 2011-0218203 (rheumatoid arthritis), U.S. Application Publication No. 2011-0217295 (active lupus arthritis), and U.S. Application Publication No. 2012-0142730 (reducing fatigue, improving quality of life, and providing neuroprotection in MS patients), each of which is hereby incorporated by reference in its entireties into this application.

A pharmaceutically acceptable salt of laquinimod as used in this application includes lithium, sodium, potassium, magnesium, calcium, manganese, copper, zinc, aluminum and iron. Salt formulations of laquinimod and the process for preparing the same are described, e.g., in U.S. Pat. No. 7,589,208 and PCT International Application Publication No. WO 2005/074899, which are hereby incorporated by reference into this application.

Laquinimod can be administered in admixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. The unit can be in a form suitable for oral administration. Laquinimod can be administered alone but is generally mixed with a pharmaceutically acceptable carrier, and co-administered in the form of a tablet or capsule, liposome, or as an agglomerated powder. Examples of suitable solid carriers include lactose, sucrose, gelatin and agar. Capsule or tablets can be easily formulated and can be made easy to swallow or chew; other solid forms include granules, and bulk powders.

Tablets may contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, dicalcium phosphate, calcium sulfate, mannitol, sorbitol, microcrystalline cellulose and the like. Suitable binders include starch, gelatin, natural sugars such as glucose or beta-lactose, corn starch, natural and synthetic gums such as acacia, tragacanth, or sodium alginate, povidone, carboxymethylcellulose, polyethylene glycol, waxes, and the like. Lubricants used in these dosage forms include sodium oleate, sodium stearate, sodium benzoate, sodium acetate, sodium chloride, stearic acid, sodium stearyl fumarate, talc and the like. Disintegrators include, without limitation, starch, methyl cellulose, agar, bentonite, xanthan gum, croscarmellose sodium, sodium starch glycolate and the like.

Specific examples of the techniques, pharmaceutically acceptable carriers and excipients that may be used to formulate oral dosage forms of the present invention are described, e.g., in U.S. Pat. No. 7,589,208, PCT International Application Publication Nos. WO 2005/074899, WO 2007/047863, and 2007/146248.

General techniques and compositions for making dosage forms useful in the present invention are described in the following references: Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds).; Modern Pharmaceutics Drugs and the Pharmaceutical Sciences, Vol. 40 (Gilbert S. Banker, Christopher T. Rhodes, Eds). These references in their entireties are hereby incorporated by reference into this application.

Disclosed is a method for treating a subject, e.g., human patient, afflicted with multiple sclerosis, e.g., relapsing multiple sclerosis or presenting a CIS using laquinimod with an anti-CD52 antibody which provides a more efficacious treatment than each agent alone. The use of laquinimod for multiple sclerosis had been previously suggested in, e.g., U.S. Pat. No. 6,077,851. However, the inventors have surprisingly found that the combination of laquinimod and an anti-CD52 antibody, e.g., alemtuzumab, specifically, the combination of the anti-CD52 antibody induction therapy and laquinimod maintenance therapy, is particularly effective for the treatment of a subject afflicted with MS or presenting a CIS as compared to each agent or each therapy alone. In particular, the anti-CD52 antibody induction therapy followed by laquinimod maintenance therapy provides substantial and sustained reduction in inflammatory activity, neuroprotection and disability reduction in part independent of inflammation.

Terms

As used herein, and unless stated otherwise, each of the following terms shall have the definition set forth below.

As used herein, “laquinimod” means laquinimod acid or a pharmaceutically acceptable salt thereof.

As used herein, an “antibody which binds to the same epitope as alemtuzumab” means an antibody which binds to the epitope of the cell surface glycoprotein CD52 to which alemtuzumab also binds.

As used herein, an “amount” or “dose” of an agent, e.g., laquinimod as measured in milligrams refers to the milligrams of the agent, e.g., laquinimod acid present in a preparation, regardless of the form of the preparation. A “dose of 0.6 mg laquinimod” means the amount of laquinimod acid in a preparation is 0.6 mg, regardless of the form of the preparation. Thus, when in the form of a salt, e.g. a laquinimod sodium salt, the weight of the salt form necessary to provide a dose of 0.6 mg laquinimod would be greater than 0.6 mg (e.g., 0.64 mg) due to the presence of the additional salt ion.

As used herein, a “unit dose”, “unit doses” and “unit dosage form(s)” mean a single drug administration entity/entities.

As used herein, “about” in the context of a numerical value or range means ±10% of the numerical value or range recited or claimed.

As used herein, a composition that is “free” of a chemical entity means that the composition contains, if at all, an amount of the chemical entity which cannot be avoided although the chemical entity is not part of the formulation and was not affirmatively added during any part of the manufacturing process. For example, a composition which is “free” of an alkalizing agent means that the alkalizing agent, if present at all, is a minority component of the composition by weight. Preferably, when a composition is “free” of a component, the composition comprises less than 0.1 wt %, 0.05 wt %, 0.02 wt %, or 0.01 wt % of the component.

As used herein, “alkalizing agent” is used interchangeably with the term “alkaline-reacting component” or “alkaline agent” and refers to any pharmaceutically acceptable excipient which neutralizes protons in, and raises the pH of, the pharmaceutical composition in which it is used.

As used herein, “oxidation reducing agent” refers to a group of chemicals which includes an “antioxidant”, a “reduction agent” and a “chelating agent”.

As used herein, “antioxidant” refers to a compound or molecule that inhibits the oxidation of other molecules. Examples of antoxidants include tocopherol, methionine, glutathione, tocotrienol, dimethyl glycine, betaine, butylated hydroxyanisole, butylated hydroxytoluene, turmerin, vitamin E, ascorbyl palmitate, tocopherol, deteroxime mesylate, methyl paraben, ethyl paraben, butylated hydroxyanisole, butylated hydroxytoluene, propyl gallate, sodium or potassium metabisulfite, sodium or potassium sulfite, alpha tocopherol or derivatives thereof, sodium ascorbate, disodium edentate, BHA (butylated hydroxyanisole), a pharmaceutically acceptable salt or ester of the mentioned compounds, and mixtures thereof.

The term “antioxidant” as used herein is also exemplified by Flavonoids such as those selected from the group of quercetin, morin, naringenin and hesperetin, taxifolin, afzelin, quercitrin, myricitrin, genistein, apigenin and biochanin A, flavone, flavopiridol, isoflavonoids such as the soy isoflavonoid, genistein, catechins such as the tea catechin epigallocatechin gallate, flavonol, epicatechin, hesperetin, chrysin, diosmin, hesperidin, luteolin, and rutin.

As used herein, “reduction agent” refers to a compound exemplified by the group consisting of thiol-containing compound, thioglycerol, mercaptoethanol, thioglycol, thiodiglycol, cysteine, thioglucose, dithiothreitol (DTT), dithio-bis-maleimidoethane (DTME), 2,6-di-tert-butyl-4-methylphenol (BHT), sodium dithionite, sodium bisulphite, formamidine sodium metabisulphite, and ammonium bisulphite.”

As used herein, “chelating agent” refers to a compound exemplified by the group consisting of penicillamine, trientine, N,N′-diethyldithiocarbamate (DDC), 2,3,2′-tetraamine (2,3,2′-tet), neocuproine, N,N,N′,N′-tetrakis(2-pyridylmethyl)ethylenediamine (TPEN), 1,10-phenanthroline (PHE), tetraethylenepentamine, triethylenetetraamine and tris(2-carboxyethyl) phosphine (TCEP), ferrioxamine, CP94, EDTA, deferoxainine B (DFO) as the methanesulfonate salt (also known as desferrioxanilne B mesylate (DFOM)), desferal from Novartis (previously Ciba-Giegy), and apoferritin.

As used herein, a pharmaceutical composition is “stable” when the composition preserves the physical stability/integrity and/or chemical stability/integrity of the active pharmaceutical ingredient during storage. Furthermore, “stable pharmaceutical composition” is characterized by its level of degradation products not exceeding 5% at 40° C./75% RH after 6 months or 3% at 55° C./75% RH after two weeks, compared to their level in time zero.

As used herein, “combination” means an assemblage of reagents for use in therapy either by simultaneous or contemporaneous administration. Simultaneous administration refers to administration of an admixture (whether a true mixture, a suspension, an emulsion or other physical combination) of a first and a second reagent. In this case, the combination may be the admixture or separate containers of the first and the second reagent that are combined just prior to administration. Contemporaneous administration refers to the separate administration of the first and the second reagent at the same time, or at times sufficiently close together that a synergistic activity relative to the activity of either the first or the second reagent alone is observed.

As used herein, “concomitant administration” or administering “concomitantly” means the administration of two agents given in close enough temporal proximately to allow the individual therapeutic effects of each agent to overlap.

As used herein, “add-on” or “add-on therapy” means an assemblage of reagents for use in therapy, wherein the subject receiving the therapy begins a first treatment regimen of one or more reagents prior to beginning a second treatment regimen of one or more different reagents in addition to the first treatment regimen, so that not all of the reagents used in the therapy are started at the same time. For example, adding laquinimod therapy to a patient already receiving alemtuzumab therapy.

As used herein, “induction therapy” means the first therapeutic measure in a series of therapeutic measures taken to treat a disease. The induction phase of the treatment (during which induction therapy is given) is frequently associated with a potent regimen and is often relatively short in duration when compared to the maintenance phase. Once the induction phase ends the maintenance phase begins during which “maintenance therapy” is given to control the disease. Alternatively, the maintenance therapy may begin any time during the induction therapy phase or any time after the completion of the induction therapy. Maintenance therapy is often a simpler and less aggressive regimen as compared to the induction therapy. In an embodiment of the present invention, the induction therapy is alemtuzumab induction therapy and induces CD52 cell depletion.

As used herein, a subject/patient who has “received an anti-CD52 antibody induction therapy” or who has “received alemtuzumab induction therapy” refers to a subject/patient who completed the induction therapy or who received at least one dose of the anti-CD52 antibody/alemtuzumab.

As used herein, “effective” when referring to an amount of laquinimod and/or anti-CD52 antibody or a therapy regimen using laquinimod and/or anti-CD52 antibody refers to the quantity or regimen of laquinimod and/or anti-CD52 antibody that is sufficient to yield a desired therapeutic response. Efficacy can be measured by an improvement of a symptom of multiple sclerosis. Such symptoms can include a MRI-monitored multiple sclerosis disease activity, relapse rate, accumulation of physical disability, frequency of relapses, time to confirmed disease progression, time to confirmed relapse, frequency of clinical exacerbation, brain atrophy, neuronal dysfunction, neuronal injury, neuronal degeneration, neuronal apoptosis, risk for confirmed progression, visual function, fatigue, impaired mobility, cognitive impairment, brain volume, abnormalities observed in whole Brain MTR histogram, general health status, functional status, quality of life, and/or symptom severity on work.

In an embodiment, an effective amount or regimen is an amount or regimen that is sufficient to reduce relapse rate, preserve brain tissue, decrease or inhibit reduction of brain volume (optionally brain volume is measured by percent brain volume change (PBVC)), increase time to confirmed disease progression (e.g., by 20-60% or at least 50%), reduce disability progression, decrease abnormalities observed in whole Brain MTR histogram, decrease the accumulation of physical disability (optionally measured by Kurtzke Expanded Disability Status Scale (EDSS) score, e.g., wherein the accumulation of physical disability is assessed by the time to confirmed disease progression as measured by Kurtzke Expanded Disability Status Scale (EDSS) score), improve impaired mobility (optionally assessed by the Timed-25 Foot Walk test, the 12-Item Multiple Sclerosis Walking Scale (MSWS-12) self-report questionnaire, the Ambulation Index (AI), the Six-Minute Walk (6MW) Test, or the Lower Extremity Manual Muscle Test (LEMMT) Test), reduce cognitive impairment (optionally assessed by the Symbol Digit Modalities Test (SDMT) score), improve general health (optionally assessed by the EuroQoL (EQ5D) questionnaire, Subject Global Impression (SGI) or Clinician Global Impression of Change (CGIC)), improve functional status (optionally measured by the subject's Short-Form General Health survey (SF-36) Subject Reported Questionnaire score), improve quality of life (optionally assessed by SF-36, EQ5D, Subject Global Impression (SGI) or Clinician Global Impression of Change (CGIC)), improve the subject's SF-36 mental component summary score (MSC) and/or SF-36 physical component summary sore (PSC), reduce level of fatigue (optionally assessed by the EQ5D, the subject's Modified Fatigue Impact Scale (MFIS) score or the French valid versions of the Fatigue Impact Scale (EMIF-SEP) score), or improve symptom severity on work (optionally measured by the work productivity and activities impairment General Health (WPAI-GH) questionnaire).

“Administering to the subject” or “administering to the (human) patient” means the giving of, dispensing of, or application of medicines, drugs, or remedies to a subject/patient to relieve, cure, or reduce the symptoms associated with a condition, e.g., a pathological condition. The administration can be periodic administration. As used herein, “periodic administration” means repeated/recurrent administration separated by a period of time. The period of time between administrations is preferably consistent from time to time. Periodic administration can include administration, e.g., once daily, twice daily, three times daily, four times daily, weekly, twice weekly, three times weekly, four times weekly and so on, etc.

“Treating” as used herein encompasses, e.g., inducing inhibition, regression, or stasis of a disease or disorder, e.g., Relapsing MS (RMS), or alleviating, lessening, suppressing, inhibiting, reducing the severity of, eliminating or substantially eliminating, or ameliorating a symptom of the disease or disorder. “Treating” as applied to patients presenting CIS can mean delaying the onset of clinically definite multiple sclerosis (CDMS), delaying the progression to CDMS, reducing the risk of conversion to CDMS, or reducing the frequency of relapse in a patient who experienced a first clinical episode consistent with multiple sclerosis and who has a high risk of developing CDMS.

“Inhibition” of disease progression or disease complication in a subject means preventing or reducing the disease progression and/or disease complication in the subject.

A “symptom” associated with MS or RMS includes any clinical or laboratory manifestation associated with MS or RMS and is not limited to what the subject can feel or observe.

As used herein, “a subject afflicted with multiple sclerosis” or “a subject afflicted with relapsing multiple sclerosis” means a subject who has been clinically diagnosed to have multiple sclerosis or relapsing multiple sclerosis (RMS), which includes relapsing-remitting multiple sclerosis (RRMS) and Secondary Progressive multiple sclerosis (SPMS).

As used herein, a subject at “baseline” is as subject prior to the first administration of laquinimod or alemtuzumab.

A “patient at risk of developing MS” (i.e. clinically definite MS) as used herein is a patient presenting any of the known risk factors for MS. The known risk factors for MS include any one of a clinically isolated syndrome (CIS), a single attack suggestive of MS without a lesion, the presence of a lesion (in any of the CNS, PNS, or myelin sheath) without a clinical attack, environmental factors (geographical location, climate, diet, toxins, sunlight), genetics (variation of genes encoding HLA-DRB1, IL7R-alpha and IL2R-alpha), and immunological components (viral infection such as by Epstein-Barr virus, high avidity CD4⁺ T cells, CD8⁺ T cells, anti-NF-L, anti-CSF 114(Glc)).

“Clinically isolated syndrome (CIS)” as used herein refers to 1) a single clinical attack (used interchangeably herein with “first clinical event” and “first demyelinating event”) suggestive of MS, which, for example, presents as an episode of optic neuritis, blurring of vision, diplopia, involuntary rapid eye movement, blindness, loss of balance, tremors, ataxia, vertigo, clumsiness of a limb, lack of co-ordination, weakness of one or more extremity, altered muscle tone, muscle stiffness, spasms, tingling, paraesthesia, burning sensations, muscle pains, facial pain, trigeminal neuralgia, stabbing sharp pains, burning tingling pain, slowing of speech, slurring of words, changes in rhythm of speech, dysphagia, fatigue, bladder problems (including urgency, frequency, incomplete emptying and incontinence), bowel problems (including constipation and loss of bowel control), impotence, diminished sexual arousal, loss of sensation, sensitivity to heat, loss of short term memory, loss of concentration, or loss of judgment or reasoning, and 2) at least one lesion suggestive of MS. In a specific example, CIS diagnosis would be based on a single clinical attack and at least 2 lesions suggestive of MS measuring 6 mm or more in diameter.

“Relapse Rate” is the number of confirmed relapses per unit time. “Annualized relapse rate” is the mean value of the number of confirmed relapses of each patient multiplied by 365 and divided by the number of days that patient is on the study drug.

“Expanded Disability Status Scale” or “EDSS” is a rating system that is frequently used for classifying and standardizing the condition of people with multiple sclerosis. The score ranges from 0.0 representing a normal neurological exam to 10.0 representing death due to MS. The score is based upon neurological testing and examination of functional systems (FS), which are areas of the central nervous system which control bodily functions. The functional systems are: Pyramidal (ability to walk), Cerebellar (coordination), Brain stem (speech and swallowing), Sensory (touch and pain), Bowel and bladder functions, Visual, Mental, and Other (includes any other neurological findings due to MS) (Kurtzke J F, 1983).

A “confirmed progression” of EDSS, or “confirmed disease progression” as measured by EDSS score is defined as a 1 point increase from baseline EDSS if baseline EDSS was between 0 and 5.0, or a 0.5 point increase if baseline EDSS was 5.5. In order to be considered a confirmed progression, the change (either 1 point or 0.5 points) must be sustained for at least 3 months. In addition, confirmation of progression cannot be made during a relapse.

“Adverse event” or “AE” means any untoward medical occurrence in a clinical trial subject administered a medicinal product and which does not have a causal relationship with the treatment. An adverse event can therefore be any unfavorable and unintended sign including an abnormal laboratory finding, symptom, or diseases temporally associated with the use of an investigational medicinal product, whether or not considered related to the investigational medicinal product.

“Gd-enhancing lesion” refers to lesions that result from a breakdown of the blood-brain barrier, which appear in contrast studies using gandolinium contrast agents. Gandolinium enhancement provides information as to the age of a lesion, as Gd-enhancing lesions typically occur within a six week period of lesion formation.

“Magnetization Transfer Imaging” or “MTI” is based on the magnetization interaction (through dipolar and/or chemical exchange) between bulk water protons and macromolecular protons. By applying an off resonance radio frequency pulse to the macromolecular protons, the saturation of these protons is then transferred to the bulk water protons. The result is a decrease in signal (the net magnetization of visible protons is reduced), depending on the magnitude of MT between tissue macromolecules and bulk water. “MT” or “Magnetization Transfer” refers to the transfer of longitudinal magnetization from the hydrogen nuclei of water that have restricted motion to the hydrogen nuclei of water that moves with many degrees of freedom. With MTI, the presence or absence of macromolecules (e.g. in membranes or brain tissue) can be seen (Mehta, 1996; Grossman, 1994).

“Magnetization Resonance Spectroscopy” or “MRS” is a specialized technique associated with magnetic resonance imaging (MRI). MRS is used to measure the levels of different metabolites in body tissues. The MR signal produces a spectrum of resonances that correspond to different molecular arrangements of the isotope being “excited”. This signature is used to diagnose certain metabolic disorders, especially those affecting the brain, (Rosen, 2007) as well as to provide information on tumor metabolism (Golder, 2007).

As used herein “mobility” refers to any ability relating to walking, walking speed, gait, strength of leg muscles, leg function and the ability to move with or without assistance. Mobility can be evaluated by one or more of several tests including but not limited to Ambulation Index, Time 25 foot walk, Six-Minute Walk (6MW), Lower Extremity Manual Muscle Test (LEMMT) and EDSS. Mobility can also be reported by the subject, for example by questionnaires, including but not limited to 12-Item Multiple Sclerosis Walking Scale (MSWS-12). Impaired Mobility refers to any impairment, difficulty or disability relating to mobility.

“T1-weighted MRI image” refers to an MR-image that emphasizes T1 contrast by which lesions may be visualized. Abnormal areas in a T1-weighted MRI image are “hypointense” and appear as dark spots. These spots are generally older lesions.

“T2-weighted MRI image” refers to an MR-image that emphasizes T2 contrast by which lesions may be visualized. T2 lesions represent new inflammatory activity.

The “Six-Minute Walk (6MW) Test” is a commonly used test developed to assess exercise capacity in patients with COPD (Guyatt, 1985). It has been used also to measure mobility in multiple sclerosis patients (Clinical Trials Website).

The “Timed-25 Foot Walk” or “T25-FW” is a quantitative mobility and leg function performance test based on a timed 25-walk. The patient is directed to one end of a clearly marked 25-foot course and is instructed to walk 25 feet as quickly as possible, but safely. The time is calculated from the initiation of the instruction to start and ends when the patient has reached the 25-foot mark. The task is immediately administered again by having the patient walk back the same distance. Patients may use assistive devices when doing this task. The score for the T25-FW is the average of the two completed trials. This score can be used individually or used as part of the MSFC composite score (National MS Society Website).

One of the central symptoms of multiple sclerosis is fatigue. Fatigue can be measured by several tests including but not limited to decrease of French valid versions of the Fatigue Impact Scale (EMIF-SEP) score, and European Quality of Life (EuroQoL) Questionnaire (EQ5D). Other tests, including but not limited to Clinician Global Impression of Change (CGIC) and Subject Global Impression (SGI), as well as EQ-5D, can be used to evaluate the general health status and quality of life of MS patients.

“Ambulation Index” or “AI” is a rating scale developed by Hauser et al. to assess mobility by evaluating the time and degree of assistance required to walk 25 feet. Scores range from 0 (asymptomatic and fully active) to 10 (bedridden). The patient is asked to walk a marked 25-foot course as quickly and safely as possible. The examiner records the time and type of assistance (e.g., cane, walker, crutches) needed. (Hauser, 1983)

“EQ-5D” is a standardized questionnaire instrument for use as a measure of health outcome applicable to a range of health conditions and treatments. It provides a simple descriptive profile and a single index value for health status that can be used in the clinical and economic evaluation of health care as well as population health surveys. EQ-5D was developed by the “EuroQoL” Group which comprises a network of international, multilingual, multidisciplinary researchers, originally from seven centers in England, Finland, the Netherlands, Norway and Sweden. The EQ-5D questionnaire is in the public domain and can be obtained from EuroQoL.

“SF-36” is a multi-purpose, short-form health survey with 36 questions which yields an 8-scale profile of functional health and well-being scores as well as psychometrically-based physical and mental health summary measures and a preference-based health utility index. It is a generic measure, as opposed to one that targets a specific age, disease, or treatment group. The survey is developed by and can be obtained from QualityMetric, Inc. of Providence, R.I.

A “pharmaceutically acceptable carrier” refers to a carrier or excipient that is suitable for use with humans and/or animals without undue adverse side effects (such as toxicity, irritation, and allergic response) commensurate with a reasonable benefit/risk ratio. It can be a pharmaceutically acceptable solvent, suspending agent or vehicle, for delivering the instant compounds to the subject.

It is understood that where a parameter range is provided, all integers within that range, and tenths thereof, are also provided by the invention. For example, “0.1-2.5 mg/day” includes 0.1 mg/day, 0.2 mg/day, 0.3 mg/day, etc. up to 2.5 mg/day.

This invention will be better understood by reference to the Experimental Details which follow, but those skilled in the art will readily appreciate that the specific experiments detailed are only illustrative of the invention as described more fully in the claims which follow thereafter.

### EXPERIMENTAL DETAILS

The mechanism of action of alemtuzumab, particular in multiple sclerosis patients, is summarized in Wiendl & Kieseier 2013, Edan 2013, Arnold 2012 and Jones 2009, each of which is hereby incorporated by reference in its entireties into this application. Alemtuzumab's effect on inflammatory axis is large and sustained. In addition, alemtuzumab exhibits well characterized depletion repletion pattern for T cells, B cells and monocytes. Further, Alemtuzumab was suggested to rebalance immune-tolerance networks including TREGS numbers and produce anti-inflammatory cytokine (Jones 2009).

Laquinimod mostly targets neurodegeneration with its primary activity in the CNS. It was thought to interfere with microglial and astrocytic activation. In MS, laquinimod showed potential for slowing neurodegeneration and reducing non-relapse-dependent disability progression. Laquinimod was also shown to sustain Confirmed Disease Progression (CDP) which was unexplained by inflammatory markers. Laquinimod also has potential for resetting immune phenotype to anti-inflammatory during reconstitution.

Treatment of MS by induction with alemtuzumab followed by maintenance with oral laquinimod leads to a substantial reduction in inflammatory activity. This treatment regimen also leads to neuroprotection and disability reduction in part independent of inflammation with laquinimod. This treatment regimen is appropriate for patients seeking maximal protection early in the disease course.

### EXAMPLE 1

### Assessment of Efficacy of Alemtuzumab Induction Therapy Followed by Laquinimod Maintenance Therapy in Multiple Sclerosis (MS) Patients

Periodic oral administration of laquinimod (p.o. 0.6 mg/day or 1.2 mg/day) as a maintenance therapy for a human patient afflicted with a form of MS who is undergoing or who has completed an induction therapy of alemtuzumab (IV 12 mg/day or 24 mg/day for at least 3 days or at least 5 days; The maintenance therapy begins on any day between Day 0 and Day 5 of the induction therapy, or any day up to Day 10 after the completion of the induction therapy) provides a clinically meaningful advantage and is more effective (provides at least an additive effect or more than an additive effect) in treating the patient than when each agent (at the same dose) is administered alone or when each therapy (at the same regimen) is employed alone.

The therapy also provides efficacy (provides at least an additive effect or more than an additive effect) in treating the patient without undue adverse side effects or affecting the safety of the treatment. As compared to when each agent or each regimen alone, alemtuzumab induction therapy followed by laquinimod maintenance therapy is:
1. more effective (provides an additive effect or more than an additive effect) in sustaining (e.g., preventing, reducing or delaying) EDSS progression in multiple sclerosis patients after receiving the maintenance therapy for 6 months.
2. more effective (provides an additive effect or more than an additive effect) in reducing the decrease in brain volume (determined by the percent brain volume change (PBVC)), in multiple sclerosis patients.
3. more effective (provides an additive effect or more than an additive effect) in increasing the time to confirmed disease progression (CDP), in multiple sclerosis patients, where CDP is defined as a sustained increase in EDSS of ≧1 point from Baseline for at least 3 months. Progression cannot be confirmed during a relapse.
4. more effective (provides an additive effect or more than an additive effect) in reducing abnormalities observed in whole Brain MTR histogram, in multiple sclerosis patients.
5. more effective (provides an additive effect or more than an additive effect) in reducing the number of confirmed relapses and therefore the relapse rate, in multiple sclerosis patients.
6. more effective (provides an additive effect of more than an additive effect) in reducing the accumulation of physical disability in multiple sclerosis patients, as measured by the time to confirmed progression of EDSS.
7. more effective (provides an additive effect or more than an additive effect) in reducing MRI-monitored disease activity in multiple sclerosis patients, as measured by the cumulative number of T1 Gd-enhancing lesions on T1-weighted images, the cumulative number new T1 hypointense lesions, the cumulative number of new T2 lesions, the cumulative number of new T1 hypointense lesions on T1-weight images (black holes), the number of active (new T2 or GdE-T1) lesions, presence or absence of GdE lesions, change in total volume of T1 Gd-enhancing lesions, change in total volume of T2 lesions, and/or cortical thickness.
8. more effective (provides an additive effect or more than an additive effect) in reducing brain atrophy in multiple sclerosis patients.
9. more effective (provides an additive effect or more than an additive effect) in reducing the frequency of relapses, the frequency of clinical exacerbation, and the risk for confirmed progression in multiple sclerosis patients.
10. more effective (provides an additive effect or more than an additive effect) in increasing the time to confirmed relapse in multiple sclerosis patients.
11. more effective (provides an additive effect or more than an additive effect) in improving the general health status (as assessed by the EuroQoL (EQ5D) questionnaire), symptom severity on work (as assessed by the work productivity and activities impairment General Health (WPAI-GH) questionnaire) and quality of life, in multiple sclerosis patients.
12. more effective (provides an additive effect or more than an additive effect) in decreasing cerebral dysfunction/cognitive impairment (as assessed by Symbol Digit Modalities Test (SDMT)), in multiple sclerosis patients during the double blind study period.

### EXAMPLE 2

### Assessment of Efficacy of Alemtuzumab Induction Therapy Followed by Laquinimod Maintenance Therapy in CIS Patients

Periodic oral administration of laquinimod (p.o. 0.6 mg/day or 1.2 mg/day) as a maintenance therapy for a human patient who is undergoing or who has completed an induction therapy of alemtuzumab (IV 12 mg/day or 24 mg/day for at least 3 days or at least 5 days; The maintenance therapy begins on any day between Day 0 and Day 5 of the induction therapy, or any day up to Day 10 after the completion of the induction therapy) provides a clinically meaningful advantage and is more effective (provides an additive effect or more than an additive effect) in delaying the conversion to clinically definite MS in patients presenting a CIS suggestive of MS than when each agent (at the same dose) is administered alone or when each therapy (at the same regimen) is employed alone.

Periodic oral administration of laquinimod (p.o. 0.6 mg/day or 1.2 mg/day) as a maintenance therapy for a human patient who is undergoing or who has completed an induction therapy of alemtuzumab (IV 12 mg/day or 24 mg/day for at least 3 days or at least 5 days) provides a clinically meaningful advantage and is more effective (provides an additive effect or more than an additive effect) in reducing the rate of development of clinically definite MS, the occurrence of new MRI-detected lesions in the brain, the accumulation of lesion area in the brain and brain atrophy in persons at high risk for developing MS, and is more effective in reducing the occurrence of clinically definite MS and preventing irreversible brain damage in these persons than when each agent (at the same dose) is administered alone or when each therapy (at the same regimen) is employed alone.

### EXAMPLE 3

### Assessment of Efficacy of CD52 Specific Antibody Induction Therapy Followed by Laquinimod Maintenance Therapy in Mice Models of Multiple Sclerosis

Laquinimod is periodically administered, as a maintenance therapy, to mice (models of MS, e.g., EAE), which have completed or are undergoing an induction therapy of a CD52 specific antibody (periodic administration for at least 3 days or at least 5 days). The maintenance therapy begins on any day between Day 0 and Day 5 of the induction therapy, or any day up to Day 10 after the completion of the induction therapy, and continues for a period of 1-96 weeks or more.

The CD52 specific antibody induction therapy followed by laquinimod maintenance therapy provides a clinically meaningful advantage and improved therapeutic efficacy (provides at least an additive effect or more than an additive effect) when compared with when each agent (at the same dose) is administered alone or when each therapy (at the same regimen) is employed alone. The therapy also provides therapeutic efficacy without undue adverse side effects or affecting the safety of the treatment.

### EXAMPLE 4

### Assessment of Efficacy of Alemtuzumab Induction Therapy Followed by Laquinimod Maintenance Therapy in Mice Models of Multiple Sclerosis

Laquinimod is periodically administered, as a maintenance therapy, to mice models of MS which have completed or are undergoing an induction therapy of alemtuzumab (periodic administration for at least 3 days or at least 5 days). The maintenance therapy begins on any day between Day 0 and Day 5 of the induction therapy, or any day up to Day 10 after the completion of the induction therapy, and continues for a period of 1-96 weeks or more.

The alemtuzumab induction therapy followed by laquinimod maintenance therapy provides a clinically meaningful advantage and improved therapeutic efficacy (provides at least an additive effect or more than an additive effect) when compared with when each agent (at the same dose) is administered alone or when each therapy (at the same regimen) is employed alone. The therapy also provides therapeutic efficacy without undue adverse side effects or affecting the safety of the treatment.

### REFERENCES

1. Barkhof, F. (1999) “MRI in Multiple Sclerosis: Correlation with Expanded Disability Status Scale (EDSS)”, Multiple Sclerosis. 5(4):283-286 (Abstract).
2. Bjartmar C and Fox R I. (2002) “Pathological mechanisms and disease progression of multiple sclerosis: therapeutic implication”, Drugs of Today. 38:7-29.
3. Brex et al., (2002) “A longitudinal study of abnormalities on MRI and disability from multiple sclerosis”, N Engl J Med. Jan. 17, 2002 346(3):158-64.
4. Brod et al. (2000) Annals of Neurology, 47:127-131.
5. Brück (2011) “Insight into the mechanism of laquinimod action.” J Neurol Sci. 2011 Jul. 15;306 (1-2):173-9.
6. Brunmark C et al., (2002) “The new orally active immunoregulator laquinimod (ABR-215062) effectively inhibits development and relapses of experimental autoimmune encephalomyelitis”, J Neuroimmunology. 130:163-172.
7. Comi et al. (2007) LAQ/5062 Study Group. “The Effect of Two Doses of Laquinimod on MRI-Monitored Disease Activity in Patients with Relapsing-Remitting Multiple Sclerosis: A Multi-Center, Randomized, Double-Blind, Placebo-Controlled Study”, Presented at: 59th Annual Meeting of the American Academy of Neurology; Apr. 28-May 5, 2007; Boston, Mass.
8. Comi et al. (2012) “Placebo-controlled trial of oral laquinimod for multiple sclerosis” N Engl J Med 2012; 2012 Mar. 15;3 66 (11):1000-9. doi: 10.1056/NEJMoa1104318.
9. Compston, Genetic susceptibility to multiple sclerosis, in McAlpine's Mutiple Sclerosis, Matthews, B. ed., London: Churchill Livingstone, 1991, 301-319.
10. Conway and Cohen (2010) “Combination therapy in multiple sclerosis”, LancetNeurol, 9:299-308.
11. Costello et al. (2007) “Combination therapies for multiple sclerosis: scientific rationale, clinical trials, and clinical practice”, Current Opinion in Neurology, 20:281-285.
12. Dal Canto et al. (1977) Multiple sclerosis. Animal model: Theiler's virus infection in mice. Am. J. Path. 88:497-500.
13. De Stefano et al. (1999) “Evidence of early axonal damage in patients with multiple sclerosis”, Neurology. 52(Suppl 2):A378.
14. Dunitz, M. Multiple sclerosis therapeutics, Ed. Rudick and Goodkin. London: Taylor & Francis, 1999.
15. Durelli et al. and the Independent Comparison of Interferon (INCOMIN) Trial Study Group. (2002) “Every-other-day interferon beta-1b versus once-weekly interferon beta-1a for multiple sclerosis: results of a 2-year prospective randomised multicentre study (INCOMIN)”, Lancet. 359:1453-60.
16. EMEA Guideline on Clinical Investigation of Medicinal Products for the Treatment of Multiple Sclerosis (CPMP/EWP/561/98 Rev. 1, November 2006).
17. EPAR, Rebif®, Scientific Discussion.
18. Fernández (2007) “Combination therapy in multiple sclerosis”, Journal of the neurological sciences, 259:95-103.
19. Fischer et al., (1999) “The Multiple Sclerosis Functional Composite measure (MSFC): an integrated approach to MS clinical outcome assessment” Multiple Sclerosis, 5(4):244-250.
20. Fisk et al., (1994) “Measuring the Functional Impact of Fatigue: Initial Validation of Fatigue Impact Scale”, Clin Inf Dis. 18 Suppl 1:S79-83.
21. Fisk et al., (1994) “The Impact of Fatigue on Patients with Multiple Sclerosis”, Can J Neurol Sci. 21:9-14.
22. Frohman et al., (2003) “The utility of MRI in suspected MS: report of the Therapeutics and Technology Assessment Subcommittee of the American Academy of Neurology”, Neurology. Sep. 9, 2003, 61(5):602-11.
23. Gold (2008) “Combination therapies in multiple sclerosis”, J Neurol, 255[Suppl 1]:51-60.
24. Golder W, (2007) “Magnetic resonance spectroscopy in clinical oncology”, Onkologie. 27(3): 304-9.
25. Grossman et al., (1994) Magnetization transfer: theory and clinical applications in neuroradiology”, RadioGraphics. 14:279-290.
26. Guidance for Industry. In vivo drug metabolism/drug interaction studies—study design, data analysis, and recommendations for dosing and labeling, U.S. Dept. Health and Human Svcs., FDA, Ctr. for Drug Eval. and Res., Ctr. For Biologies Eval. and Res., Clin./Pharm., November 1999 <http://www.fda.gov/cber/gdlns/metabol.pdf>.
27. Gurevich et al. (2010) “Laquinimod suppress antigen presentation in relapsing-remitting multiple sclerosis: in vitro high-throughput gene expression study” (J Neuroimmunol. 2010 Apr. 15;221 (1-2):87-94. Epub 2010 Mar. 27.
28. Guyatt et al. (1985) “The 6-minute walk: a new measure of exercise capacity in patients with chronic heart failure”, Can Med Assoc J., 132:919-823.
29. Hafler and Weiner, M S: A CNS and systemic autoimmune disease, Immunol. Today, 1989, 10:104-107.
30. Hartung et al. (2005) “Significance of neutralizing antibodies to interferon beta during treatment of multiple sclerosis: expert opinions based on the Proceedings of an International Consensus Conference”, Eur J Neurol. 12:588-601.
31. Hauser et al. (1983) “Intensive immunosuppression in progressive multiple sclerosis”, New Engl J Med. 308:173-180.
32. Hohlfeld et al. (2000) “The neuroprotective effect of inflammation: implications for the therapy of multiple sclerosis”, J Neuroimmunol. 107:161-166.
33. Kleinschmidt-DeMasters et al. (2005) New England Journal of Medicine, 353:369-379.
34. Kurtzke J F, (1983) “Rating neurologic impairment in multiple sclerosis: an expanded disability status scale (EDSS)”, Neurology 33(11):1444-1452.
35. Lampert, Autoimmune and virus-induced demyelinating diseases. A review, Am. J. Path., 1978, 91:176-208.
36. Langer-Gould et al. (2005) New England Journal of Medicine, 353:369-379.
37. Lublin F D, Reingold S C (1996) “Defining the clinical course of multiple sclerosis”, Neurol. 46:907-911.
38. Martyn, The epidemiology of multiple sclerosis, in McAlpine's Multiple Sclerosis, Matthews, B., ed., London: Churchil Livingstone, 1991, 3-40.
39. McDonald et al., Recommended diagnostic criteria for multiple sclerosis: guidelines from the International Panel on the diagnosis of multiple sclerosis. Ann. Neurol., 2001, 50:121-127.
40. McDonald, (2001) “Guidelines from the International Panel on the Diagnosis of Multiple Sclerosis” Ann. Neurol. 50:121-127.
41. Mehta et al. (1996) “Magnetization transfer magnetic resonance imaging: a clinical review”, Topics in Magnetic Resonance Imaging 8(4):214-30.
42. Miki et al. (1999) “Relapsing-Remitting Multiple Sclerosis; Longitudinal Analysis of MR Images—Lack of Correlation between Changes in T2 Lesion Volume and Clinical Findings”, Radiology. 213:395-399.
43. Milo and Panitch (2011) “Combination therapy in multiple sclerosis”, Journal of Neuroimmunology, 231(2011):23-31.
44. Moraal et al. (2008) “Subtraction MR Images in a Multiple Sclerosis Multicenter Clinical Trial Setting”, Radiology, 250(2):506-514.
45. Multiple sclerosis: its diagnosis, symptoms, types and stages, 2003 <http://www.albany.net/-tjc/multiple-sclerosis.html>.
46. National MS Society Website, retrieved Jul. 10, 2012 <http://www.nationalmssociety.org/ms-clinical-care-network/researchers/clinical-study-measures/index.aspx>
47. Neuhaus et al. (2003) “Immunomodulation in multiple sclerosis: from immunosuppression to neuroprotection”, Trends Pharmacol Sci. 24:131-138.
48. Noseworthy et al. (2000) “Multiple sclerosis”, N Engl J Med. 343:938-952.
49. Olsson, Immunology of multiple sclerosis, Curr. Opin. Neurol. Neurosurg., 1992, 5:195-202.
50. Panitch et al. for the EVIDENCE (Evidence of Interferon Dose-response: European North American Comparative Efficacy) Study Group and the University of British Columbia MS/MRI Research Group. (2002) “Randomized comparative study of interferon β-1a treatment regiments in MS”, The EVIDENCE Trial. Neurology. 59:1496-1506.
51. Parkman, Graft-versus-host Disease, Ann. Rev. Med., 1991, 42:189-197.
52. PCT International Application Publication No. WO 1998/30227, published Jul. 16, 1998.
53. PCT International Application Publication No. WO 2000/05250, published Feb. 3, 2000.
54. PCT International Application Publication No. WO 2000/18794, published Apr. 6, 2000.
55. PCT International Application Publication No. WO 2003/048735, published Jun. 12, 2003.
56. PCT International Application Publication No. WO 2004/103297, published Dec. 2, 2004.
57. PCT International Application Publication No. WO. 2006/016036, published Nov. 2, 2006.
58. PCT International Application Publication No. WO 2006/029393, published Mar. 16, 2006.
59. PCT International Application Publication No. WO 2006/029411, published Mar. 16, 2006.
60. PCT International Application Publication No. WO 2006/083608, published Aug. 10, 2006.
61. PCT International Application Publication No. WO 2006/089164, published Aug. 24, 2006.
62. PCT International Application Publication No. WO 2006/116602, published Nov. 2, 2006.
63. PCT International Application Publication No. WO 2007/047863, published Apr. 26, 2007.
64. PCT International Application Publication No. WO 2007/146248, published Dec. 21, 2007, international filing date Jun. 12, 2007.
65. PCT International Application Publication No. WO 2009/070298, published Jun. 4, 2009.
66. PCT International Application Publication No. WO 2011/008274, published Jan. 20, 2011.
67. PCT International Application Publication No. WO 2011/022063, published Feb. 24, 2011.
68. PCT International Application Publication No. WO 2012/051106, published Apr. 19, 2012.
69. Polman et al. (2011) “Diagnostic Criteria for Multiple Sclerosis: 2010 Revisions to the McDonald Criteria”, Ann N eural, 69:292-302.
70. Polman et al., (2005) “Diagnostic criteria for multiple sclerosis: 2005 revisions to the McDonald Criteria”, Annals of Neurology, Volume 58 Issue 6, Pages 840-846.
71. Polman et al., (2005) “Treatment with laquinimod reduces development of active MRI lesions in relapsing MS”, Neurology. 64:987-991.
72. Poser et al. (1983) “New Diagnostic Criteria for Multiple Sclerosis: Guidelines for Research Protocols”, Annals of Neurology, March 1983, 13(3):227-230.
73. Rodriguez et al. (1987) Theiler's murine encephalomyelitis: a model of demyelination and persistence of virus. Crit. Rev. Immunol., 7:325.
74. Rosen Y, (2007) “The Recent advances in magnetic resonance neurospectroscopy”, Neurotherapeutics. 27 (3):330-45.
75. RTT News Article dated Apr. 12, 11, entitled “Teva Pharma, Active Biotech Post Positive Laquinimod Phase 3 ALLEGRO Results”.
76. Rudick et al. (2006) New England Journal of Medicine, 354:911-923.
77. Rudick, R. (1999) “Disease-Modifying Drugs for Relapsing-Remitting Multiple Sclerosis and Future Directions for Multiple Sclerosis Therapeutics”, Neurotherpatueics. 56:1079-1084.
78. Runström et al. (2002) “Laquinimod (ABR-215062) a candidate drug for treatment of Multiple Sclerosis inhibits the development of experimental autoimmune encephalomyelitis in IFN-β knock-out mice”, (Abstract), Medicon Valley Academy, Malmoe, Sweden.
79. Runström et al. (2006) “Inhibition of the development of chronic experimental autoimmune encephalomyelitis by laquinimod (ABR-2150 62) in IFN-β k.o. and wild type mice” Journal of Neuroimmunology, 173 (2006):69-78.
80. Salama et al. (2003) Multiple Sclerosis, 9:28-31.
81. Sandberg-Wollheim et al. (2005) “48-week open safety study with high-dose oral laquinimod in patients”, Mult Scler. 11:S154 (Abstract).
82. Sorenson P S. (2006) “Neutralising antibodies to interferon-β—measurement, clinical relevance, and management”, J Neurol. 253 [Suppl 6]:VI/16-VI/22.
83. Teitelbaum et al., Suppression of Experimental Allergic Encephalomyelitis by a Synthetic Polypeptide, Eur. J. Immunol., 1971, 1: 242-248.
84. Teitelbaum et al., Suppression of Experimental Allergic Encephalomyelitis with Basic Polymers, Eur. J. Immunol., 1973, 3: 273-279.
85. Teva Press Release dated Aug. 1, 2011, entitled “Results of Phase III BRAVO Trial Reinforce Unique Profile of Laquinimod for Multiple Sclerosis Treatment”.
86. The National MS Society (USA), The Disease Modifying Drug Brochure, Oct. 19, 2006.
87. U.S. Patent Application Publication No. 2008-0207526, published Aug. 28, 2008 (Strominger et al.).
88. U.S. Patent Application Publication No. 2008-0279952, published Nov. 13, 2008 (Bernd Terhaag and Pirna).
89. U.S. Patent Application Publication No. 2010-0322900, published Dec. 23, 2010 (Nora Tarcic and Modiin).
90. U.S. Patent Application Publication No. 2011-0027219, published Feb. 3, 2011 (Tarcic et al.).
91. U.S. Patent Application Publication No. 2011-0034508, published Feb. 10, 2011 (Liat Hayardeny).
92. U.S. Patent Application Publication No. 2011-0217295, published Sep. 8, 2011 (Haviv and Tarcic).
93. U.S. Patent Application Publication No. 2011-0218179, published Sep. 8, 2011 (Haviv and Tarcic).
94. U.S. Patent Application Publication No. 2011-0218203, published Sep. 8, 2011 (Joel Kaye et al.).
95. U.S. Patent Application Publication No. 2011-0230413, published Sep. 22, 2011 (Suhayl Dhib-Jalbut).
96. U.S. Patent Application Publication No. 2012-0010238, published Jan. 12, 2012 (Fristedt).
97. U.S. Patent Application Publication No. 2012-0010239, published Jan. 12, 2012 (Piryatinsky et al.).
98. U.S. Patent Application Publication No. 2012-0142730, published Jun. 7, 2012 (Tarcic et al.).
99. U.S. Pat. No. 3,849,550, issued Nov. 19, 1974 (Teitelbaum et al).
100. U.S. Pat. No. 5,800,808, issued Sep. 1, 1998 (Konfino et al).
101. U.S. Pat. No. 5,858,964, issued Jan. 12, 1999 (Aharoni et al).
102. U.S. Pat. No. 5,981,589, issued Nov. 9, 1999 (Konfino et al).
103. U.S. Pat. No. 6,048,898, issued Apr. 11, 2000 (Konfino et al).
104. U.S. Pat. No. 6,054,430, issued Apr. 25, 2000 (Konfino et al).
105. U.S. Pat. No. 6,077,851, issued Jun 20, 2000 (Bjork et al).
106. U.S. Pat. No. 6,214,791, issued Apr. 10, 2001 (Arnon et al).
107. U.S. Pat. No. 6,342,476, issued Jan. 29, 2002 (Konfino et al).
108. U.S. Pat. No. 6,362,161, issued Mar. 26, 2002 (Konfino et al).
109. U.S. Pat. No. 6,794,410, issued Sep. 21, 2004 (Wettstein).
110. U.S. Pat. No. 7,566,767, issued Jul. 28, 2009 (Strominger et al.).
111. U.S. Pat. No. 7,589,208, issued Sep. 15, 2009 (Jansson et al).
112. U.S. Pat. no. 7,884,208, issued Feb. 8, 2011 (Frenkel et al.).
113. U.S. Pat. No. 7,989,473, issued Aug. 2, 2011 (Patashnik et al.).
114. U.S. Pat. No. 8,008,258, issued Aug. 30, 2011 (Aharoni et al).
115. U.S. Pat. No. 8,178,127, issued May 15, 2012 (Safadi et al.).
116. Vollmer et al. (2008) “Glatiramer acetate after induction therapy with mitoxantrone in relapsing multiple sclerosis” Multiple Sclerosis, 00:1-8.
117. Yang et al., (2004) “Laquinimod (ABR-215062) suppresses the development of experimental autoimmune encephalomyelitis, modulates the Th1/Th2 balance and induces the Th3 cytokine TGF-β in Lewis rats”, J. Neuroimmunol. 156:3-9.
118. Zou et al. (2002) “Suppression of experimental autoimmune neuritis by ABR-215062 is associated with altered Th1/Th2 balance and inhibited migration of inflammatory cells into the peripheral nerve tissue”, Neuropharmacology. 42:731.
119. Anderson (2013) “Alemtuzumab Benefits Hard-to-Treat MS Patients,” Medscape Medical News>Conference News, Jun. 2, 2013, retrieved Aug. 1, 2013 <http://www.medscape.com/viewarticle/805173>.
120. Arnold (2012) ECTRIMS 20212 CAREMS II.
121. Buttman and Rieckmann (2008) “Treating Multiple Sclerosis With Monoclonal Antibodies,” Expert Rev Neurother. 2008; 8(3):433-455.
122. ClinicalTrials.gov Web “Comparison of Alemtuzumab and Rebif(c) Efficacy in Multiple Sclerosis, Study One (CARE-MS I),” Retrieved Aug. 1, 2013 <http://clinicaltrials.gov/ct2/show/NCT00530348>.
123. ClinicalTrials.gov Web “Comparison of Alemtuzumab and Rebif® Efficacy in Multiple Sclerosis, Study Two (CARE-MS IT), “Retrieved Aug. 1, 2013 <http://clinicaltrials.gov/ct.2/show/NCT00548405>.
124. Cohen et al. (2012) “Alemtuzumab versus interferon beta 1a as first-line treatment for patients with relapsing-remitting multiple sclerosis: a randomized controlled phase 3 trial,” The Lancet, 380 (9856):1819-1828.
125. Coles A J (2013) “Alemtuzumab therapy for multiple sclerosis,” Neurotherapeutics. 2013 January; 10(1):29-33. doi: 10.1007/s13311-012-0159-0.
126. Coles et al. (2012) “Alemtuzumab for patients with relapsing multiple sclerosis after disease-modifying therapy: a randomized controlled phase 3 trial,” The Lancet, 380(9856):1829-1839.
127. Edan G and Le Page E. (2013) “Induction therapy for patients with multiple sclerosis: why? When? How?” CNS Drugs. 2013 June; 27(6):403-9.
128. Jones J, et al. (2009) “IL-21 drives secondary autoimmunity in patients with multiple sclerosis, following therapeutic lymphocyte depletion with alemtuzumab (Campath-1H),” J Clin Invest. 2009; 119(7):2052-2061. doi:10.1172/JCI37878.
129. McKee (2012) “Sanofi withdraws Campath in US and EU,” Pharma Times, Aug. 21, 2012, retrieved Aug. 1, 2013 <http://www.pharmatimes.com/article/12-08-21/Sanofi withdraws Campath in US and EU.aspx>.
130. Wiendl & Kieseier (2013) “Multiple sclerosis: reprogramming the immune repertoire with alemtuzumab in MS,” Nat Rev Neurol. 2013 March; 9(3):125-6. doi: 10.1038/nrneurol.2013.2.
131. U.S. Application Publication No. 2013-0108625, published May 2, 2013 (Margolin et al.).
132. U.S. Pat. No. 5,846,534, issued Dec. 8, 1998 to Waldmann et al.
133. U.S. Pat. No. 6,120,766, issued Sep. 19, 2000 to Hale et al.
134. European Application Publication No. EP2433649 A2, published Mar. 28, 2012 (Alcafleu Management GmbH & Co. KG).
135. European Application Publication No. EP2066352 A1, published Jun. 10, 2009 (Bayer Schering Pharma).
136. European Application Publication No. EP2444104 A3, published Oct 24, 2012 (Alcafleu Management GmbH & Co. KG).

What is claimed is:

1. A method of treating a subject afflicted with multiple sclerosis or presenting a clinically isolated syndrome and who has received an anti-CD52 antibody induction therapy, comprising periodically administering to the subject an amount of laquinimod, thereby treating the subject afflicted with multiple sclerosis or presenting a clinically isolated syndrome.

2. The method of claim 1, further comprising a step of determining that the subject has received the anti-CD52 antibody induction therapy.

3. A method of treating a subject afflicted with multiple sclerosis or presenting a clinically isolated syndrome comprising
a) administering to the subject an amount of an anti-CD52 antibody, followed by
b) periodically administering to the subject an amount of laquinimod,
thereby treating the subject afflicted with multiple sclerosis or presenting a clinically isolated syndrome.

4. The method of any one of claims 1-3, wherein the multiple sclerosis is relapsing multiple sclerosis or relapsing-remitting multiple sclerosis.

5. The method of claims 3 or 4, wherein step a) comprises periodically administering to the subject an amount of the anti-CD52 antibody.

6. The method of any one of claims 3-5, wherein step a) followed by step b) is more effective to treat the subject then step a) alone or step b) alone.

7. The method of any one of claims 3-6, wherein step a) followed by step b) is effective to reduce a symptom of multiple sclerosis in the subject.

8. The method of claim 7, wherein the symptom is a MRI-monitored multiple sclerosis disease activity, relapse rate, accumulation of physical disability, frequency of relapses, decreased time to confirmed disease progression, decreased time to confirmed relapse, frequency of clinical exacerbation, brain atrophy, neuronal dysfunction, neuronal injury, neuronal degeneration, neuronal apoptosis, risk for confirmed progression, deterioration of visual function, fatigue, impaired mobility, cognitive impairment, reduction of brain volume, abnormalities observed in whole Brain MTR histogram, deterioration in general health status, functional status, quality of life, and/or symptom severity on work.

9. The method of claim 8, wherein step a) followed by step b) is effective to
a) decrease or inhibit reduction of brain volume, and/or
b) increase time to confirmed disease progression, and/or
c) decrease abnormalities observed in whole Brain MTR histogram, and/or
d) reduce cognitive impairment.

10. The method of claim 9, wherein
a) brain volume is measured by percent brain volume change (PBVC), and/or
b) time to confirmed disease progression is increased by 20-60%, and/or
c) cognitive impairment is assessed by the Symbol Digit Modalities Test (SDMT) score.

11. The method of claim 8, wherein:
a) the accumulation of physical disability is measured by Kurtzke Expanded Disability Status Scale (EDSS) score or by the time to confirmed disease progression as measured by EDSS score, and/or
b) impaired mobility is assessed by the Timed-25 Foot Walk test, the 12-Item Multiple Sclerosis Walking Scale (MSWS-12) self-report questionnaire, the Ambulation Index (AI), the Six-Minute Walk (6MW) Test, or the Lower Extremity Manual Muscle Test (LEMMT) Test, and/or
c) general health status is assessed by the EuroQoL (EQ5D) questionnaire, Subject Global Impression (SGI) or Clinician Global Impression of Change (CGIC), and/or
d) functional status is measured by the subject's Short-Form General Health survey (SF-36) Subject Reported Questionnaire score, and/or
e) quality of life is assessed by SF-36, EQ5D, Subject Global Impression (SGI) or Clinician Global Impression of Change (CGIC), and/or
f) fatigue is assessed by the EQ5D, the subject's Modified Fatigue Impact Scale (MFIS) score or the French valid versions of the Fatigue Impact Scale (EMIF-SEP) score, and/or
g) symptom severity on work is measured by the work productivity and activities impairment General Health (WPAI-GH) questionnaire.

12. The method of claim 11, wherein the subject had an EDSS score of 0-5.5, of 1.5-4. 5, or of 5.5 or greater at baseline. cm 13. The method of claims 11 or 12, wherein confirmed disease progression is a 1 point or a 0.5 point increase of the EDSS score.

14. The method of claim 11, wherein the subject's SF-36 mental component summary score (MSC) is improved and/or wherein the subject's SF-36 physical component summary sore (PSC) is improved.

15. The method of any one of claims 1-14, wherein laquinimod is laquinimod sodium.

16. The method of any one of claims 1-15, wherein the laquinimod is administered via oral administration.

17. The method of any one of claims 1-16, wherein the anti-CD52 antibody is or was administered via injection or via intravenous injection.

18. The method of any one of claims 1-17, wherein the laquinimod and/or the anti-CD52 antibody is administered daily, more often than once daily, or less often than once daily.

19. The method of any one of claims 1-18, wherein the amount laquinimod administered is 0.1-40.0 mg/day, 0.1-2.5 mg/day, 0.25-2.0 mg/day, 0.5-1.2 mg/day or less than 0.6 mg/day. 20.The method of claim 19, wherein the amount laquinimod administered is 0.25 mg/day, 0.3 mg/day, 0.5 mg/day, 0.6 mg/day, 1.0 mg/day, 1.2 mg/day, 1.5 mg/day, or 2.0 mg/day.

21. The method of any one of claims 1-20, wherein step b) begins after completion of step a), on the same day as initiation of step a), or 1-10 days after initiation of step a).

22. The method of any of claims 3-21, further comprising:
a) administering to the subject an amount of the anti-CD52 antibody,
wherein step c) begins at least 6 months after initiation of step a).

23. The method of claim 22, wherein step c) begins after completion of step a), on the same day as initiation of step b), at least 6 months after initiation of step a), or at least 12 months after initiation of step a).

24. The method of any one of claims 1-23, wherein the amount of the anti-CD52 antibody administered in step a) and/or step c) is 3-48 mg/day, 9-36 mg/day, 12 mg/day, or 24 mg/day.

25. The method of any one of claims 3-24, wherein step a) and/or step c) comprises
b) daily administration of an amount of the anti-CD52 antibody to the subject over 1-5 days, and/or
c) at least one administration of an amount of the anti-CD52 antibody to the subject over one day, and/or
d) daily administration of an amount of the anti-CD52 antibody to the subject for at least 3 consecutive days or for at least 5 consecutive days.

26. The method of any one of claims 1-25, further comprising administration of nonsteroidal anti-inflammatory drugs (NSAIDs), salicylates, slow-acting drugs, gold compounds, hydroxychloroquine, sulfasalazine, combinations of slow-acting drugs, corticosteroids, cytotoxic drugs, immunosuppressive drugs and/or antibodies.

27. The method of any one of claims 1-26, wherein the periodic administration of laquinimod continues for at least 3 days, for more than 30 days, for more than 42 days, for 8 weeks or more, for at least 12 weeks, for at least 24 weeks, for at least 24 weeks, or for 6 months or more.

28. The method of any one of claims 3-27, wherein step a) followed by step b) inhibits a symptom of relapsing multiple sclerosis by at least 20%, by at least 30%, by at least 50%, by at least 70%, by more than 100%, by more than 300%, or by more than 1000%.

29. The method of any one of claims 3-28, wherein each of the amount of laquinimod or pharmaceutically acceptable salt thereof when taken alone, and the amount of the anti-CD52 antibody when taken alone is effective to treat the subject, or wherein either the amount of laquinimod or pharmaceutically acceptable salt thereof when taken alone, the amount of the anti-CD52 antibody when taken alone, or each such amount when taken alone is not effective to treat the subject.

30. The method of any one of claims 1-29, wherein the subject is a human patient.

31. A package comprising:
a) a first pharmaceutical composition comprising an amount of laquinimod and a pharmaceutically acceptable carrier;
b) a second pharmaceutical composition comprising an amount of an anti-CD52 antibody and a pharmaceutically acceptable carrier; and
c) instructions for use of the first and second pharmaceutical compositions together to treat a subject afflicted with multiple sclerosis or presenting a clinically isolated syndrome.

32. The package of claim 31, wherein
a) the first pharmaceutical composition, the second pharmaceutical composition, or both the first and the second pharmaceutical composition are in an aerosol, an inhalable powder, an injectable, a liquid, a solid, a capsule or a tablet form, and/or
b) the first pharmaceutical composition, the second pharmaceutical composition, or both the first and the second pharmaceutical composition are in a liquid or a solid form, and/or
c) the first pharmaceutical composition is in capsule form or in tablet form.

33. The package of claim 32, wherein the tablets are coated with a coating which inhibits oxygen from contacting the core.

34. The package of claim 33, wherein the coating comprises a cellulosic polymer, a detackifier, a gloss enhancer, or pigment.

35. The package of any one of claims 31-34, wherein the first pharmaceutical composition further comprises one, two, three or four of a)-e):
a) mannitol,
b) an alkalinizing agent,
c) an oxidation reducing agent,
d) a lubricant, and
e) a filler,
or wherein the first pharmaceutical composition further comprises each of a)-e).

36. The package of claim 35, wherein:
a) the alkalinizing agent is meglumine, and/or
b) the lubricant is present in the composition as solid particles, and/or
c) the lubricant is sodium stearyl fumarate or magnesium stearate, and/or
d) the filler is present in the composition as solid particles, and/or
e) the filler is lactose, lactose monohydrate, starch, isomalt, mannitol, sodium starch glycolate, sorbitol, lactose spray dried, lactose anhydrouse, or a combination thereof, and/or
f) the filler is mannitol or lactose monohydrate.

37. The package of any one of claims 31-36, wherein the first pharmaceutical composition is stable and free of an alkalinizing agent or an oxidation reducing agent, or wherein the first pharmaceutical composition is free of an alkalinizing agent and free of an oxidation reducing agent.

38. The package of any one of claims 31-37, wherein the first pharmaceutical composition is stable and free of disintegrant.

39. The package of any one of claims 31-38, further comprising a desiccant.

40. The package of claim 39, wherein the desiccant is silica gel.

41. The package of any one of claims 31-40, wherein the first pharmaceutical composition is stable and has a moisture content of no more than 4%.

42. The package of any one of claims 31-41, wherein laquinimod is present in the composition as solid particles.

43. The package of any one of claims 31-42, wherein the package is a sealed packaging having a moisture permeability of not more than 15 mg/day per liter. 44. The package of claim 43, wherein the sealed package is a blister pack in which the maximum moisture permeability is no more than 0.005 mg/day, or the sealed package is a bottle.

45. The package of claim 44, wherein the bottle is closed with a heat induction liner.

46. The package of any one of claims 43-45, wherein the sealed package comprises an HDPE bottle and/or an oxygen absorbing agent.

47. The package of claim 46, wherein the oxygen absorbing agent is iron.

48. The package of any one of claims 31-47, wherein the amount of laquinimod in the first composition comprises a unit dose of less than 0.6 mg, a unit dose of 0.1-40.0 mg, a unit dose of 0.1-2.5 mg, a unit dose of 0.25-2.0 mg, or a unit dose of 0.5-1.2 mg.

49. The package of claim 48, wherein the amount of laquinimod in the first composition comprises a unit dose of 0.25 mg, 0.3 mg, 0.5 mg, 0.6 mg, 1.0 mg, 1.2 mg, 1.5 mg; or 2.0 mg.

50. The package of any one of claim 31-49, wherein the amount of the anti-CD52 antibody in the second composition comprises a unit dose of 3-48 mg, a unit dose of 9-36 mg, a unit dose of 12 mg or a unit dose of 24 mg.

51. Laquinimod for use as a maintenance therapy in treating a subject afflicted with multiple sclerosis or presenting a clinically isolated syndrome, wherein the subject has received an anti-CD52 antibody induction therapy.

52. A pharmaceutical composition comprising an amount of laquinimod for use as a maintenance therapy in treating a subject afflicted with multiple sclerosis or presenting a clinically isolated syndrome, wherein the subject has received an anti-CD52 antibody induction therapy.

53. The pharmaceutical composition of claim 52, wherein laquinimod is laquinimod sodium.

54. The pharmaceutical composition of claims 52 or 53, wherein the pharmaceutical composition is:
a) in an aerosol, an inhalable powder, an injectable, a liquid, a solid, a capsule or a tablet form, and/or
b) in a liquid or a solid form, and/or
c) in capsule form or in tablet form.

55. The pharmaceutical composition of claim 54, wherein the tablets are coated with a coating which inhibits oxygen from contacting the core.

56. The pharmaceutical composition of claim 55, wherein the coating comprises a cellulosic polymer, a detackifier, a gloss enhancer, or pigment.

57. The pharmaceutical composition of any one of claims 52-56, further comprising one, two, three or four of a)-e):
a) mannitol,
b) an alkalinizing agent,
c) an oxidation reducing agent,
d) a lubricant, and
e) a filler,
or wherein the first pharmaceutical composition further comprises each of a)-e).

58. The pharmaceutical composition of claim 57, wherein:
a) the alkalinizing agent is meglumine, and/or
b) the lubricant is present in the composition as solid particles, and/or
c) the lubricant is sodium stearyl fumarate or magnesium stearate, and/or
d) the filler is present in the composition as solid particles, and/or
e) the filler is lactose, lactose monohydrate, starch, isomalt, mannitol, sodium starch glycolate, sorbitol, lactose spray dried, lactose anhydrouse, or a combination thereof, and/or
f) the filler is mannitol or lactose monohydrate.

59. The pharmaceutical composition of any one of claims 52-58, which is free of an alkalinizing agent or an oxidation reducing agent, or which is free of an alkalinizing agent and free of an oxidation reducing agent.

60. The pharmaceutical composition of any one of claims 52-59, which is stable and free of disintegrant.

61. The pharmaceutical composition of any one of claims 52-60, wherein the amount of laquinimod in the composition is 0.1-40.0 mg, 0.1-2.5 mg, 0.25-2.0 mg, 0.5-1.2 mg, or less than 0.6 mg.

62. The pharmaceutical composition of claim 61, wherein the amount of laquinimod in the composition is 0.25 mg, 0.3 mg, 0.5 mg, 0.6 mg, 1.0 mg, 1.2 mg, 1.5 mg, or 2.0 mg.

63. A pharmaceutical composition comprising an amount of laquinimod for use in treating a subject afflicted with multiple sclerosis or presenting a clinically isolated syndrome as a maintenance therapy in combination with an anti-CD52 antibody induction therapy.

64. A pharmaceutical composition comprising an amount of an anti-CD52 antibody for use in treating a subject afflicted with multiple sclerosis or presenting a clinically isolated syndrome as an induction therapy in combination with laquinimod maintenance therapy.

65. A therapeutic package for dispensing to, or for use in dispensing to, a subject afflicted with multiple sclerosis or presenting a clinically isolated syndrome, which comprises:
a) one or more unit doses, each such unit dose comprising:
i) an amount of laquinimod and/or
ii) an amount of an anti-CD52 antibody, and
b) a finished pharmaceutical container therefor, said container containing said unit dose or unit doses, said container further containing or comprising labeling directing the use of said package in the treatment of said subject.

66. The therapeutic package of claim 65, wherein the respective amounts of said laquinimod and said anti-CD52 antibody in said unit dose when taken together is more effective to treat the subject than when compared to the administration of said laquinimod in the absence of said anti-CD52 antibody or the administration of said anti-CD52 antibody in the absence of said laquinimod.

67. The method of any one of claims 1-30, the package of any one of claims 31-50, the use of claim 51, the pharmaceutical composition of any one of claims 52-64, or the therapeutic package of claims 65 or 66, wherein the anti-CD52 antibody binds to the same epitope as alemtuzumab.

68. The method of any one of claims 1-30, the package of any one of claims 1-30, the use of claim 51, the pharmaceutical composition of any one of claims 52-64, or the therapeutic package of claims 65 or 66, wherein the anti-CD52 antibody is alemtuzumab.
